SEQUENCE LISTING (15 sequences)

SEQ ID NO: 1 - protein, 30 aa, Homo sapiens
KQADYVPSDQDLLRCCVLTSGIFETKFQVD

SEQ ID NO: 2 - protein, 30 aa, Homo sapiens
KQADYVPSDQDLLRCHVLTSGIFETKFQVD

SEQ ID NO: 3 - protein, 13 aa, Homo sapiens
QDLLRCCVLTSGI

SEQ ID NO: 4 - protein, 13 aa, Homo sapiens
QDLLRCHVLTSGI

SEQ ID NO: 5 - protein, 19 aa, Homo sapiens
PSDQDLLRCCVLTSGIFET

SEQ ID NO: 6 - protein, 19 aa, Homo sapiens
PSDQDLLRCHVLTSGIFET

SEQ ID NO: 7 - protein, 25 aa, Homo sapiens
DYVPSDQDLLRCCVLTSGIFETKFQ

SEQ ID NO: 8 - protein, 25 aa, Homo sapiens
DYVPSDQDLLRCHVLTSGIFETKFQ

SEQ ID NO: 9 - protein, 30 aa, Homo sapiens
KQADYVPSDQDLLRCRVLTSGIFETKFQVD

SEQ ID NO: 10 - protein, 394 aa, Homo sapiens
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 11 - protein, 394 aa, Homo sapiens
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCCVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 12 - protein, 394 aa, Homo sapiens
MGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCHVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 13 - protein, 13 aa, Homo sapiens
DQDLLRCCVLTSG

SEQ ID NO: 14 - protein, 14 aa, Homo sapiens
QDLLRCHVLTSGIF

SEQ ID NO: 15 - protein, 12 aa, Homo sapiens
LLRCHVLTSGIF

(19) United States
(12) Patent Application Publication
(10) Pub. No.: US 2024/0287149 A1
(43) Pub. Date: Aug. 29, 2024

(54) MUTATED GNAS PEPTIDES

(71) Applicant: Oslo University Hospital, Oslo (NO)

(72) Inventors: Else Marit INDERBERG, Oslo (NO); Kjersti FLATMARK, Oslo (NO); Jon Amund ERIKSEN, Porsgrunn (NO)

(73) Assignee: Oslo University Hospital, Oslo (NO)

(21) Appl. No.: 18/560,340

(22) PCT Filed: May 13, 2022
(86) PCT No.: PCT/EP2022/063081

§ 371 Date: Nov. 10, 2023

(30) Foreign Application Priority Data
May 14, 2021 (EP) 21173961.0

(51) Int. Cl. C07K 14/47; A61K 39/00; C12N 5/0783

(52) U.S. Cl. CPC C07K 14/4748; A61K 39/0011; A61K 39/4611; A61K 39/4644; C12N 5/0636

(57) ABSTRACT

A peptide mixture suitable for eliciting an immune response, comprising a first peptide and a second peptide. The first peptide comprises a first sequence of at least (8) amino acids, wherein said first sequence has at least 75% sequence identity to a first region of (8) amino acids of SEQ ID NO: 11. This first region includes position (201) of the SEQ ID NO: 11 such that the first sequence includes the amino acid in position (201) of the SEQ ID NO: 11. The second peptide comprises a second sequence of at least (8) amino acids, wherein said second sequence has at least 75% sequence identity to a second region of (8) amino acids of SEQ ID NO: 12. This second region includes position (201) of the SEQ ID NO: 12 such that the second sequence includes the amino acid in position (201) of the SEQ ID NO: 12.

### FIELD OF THE INVENTION

The present invention provides peptide mixtures comprising mutated peptides of the GNAS protein for eliciting an immune response, mutated peptides of the GNAS protein for eliciting an immune response, a nucleic acid molecule and at least one nucleic acid molecule encoding the peptide or peptide mixture, and T-cell mixtures and T-cell preparations comprising T-cells specific for such peptides when presented on MHC molecules. The invention also relates to pharmaceutical formulations comprising such peptide mixtures and T-cell mixtures and preparations, uses of such peptide mixtures, peptides and T-cell mixtures and preparations for the prophylaxis and/or treatment of cancer.

### BACKGROUND OF THE INVENTION

Pseudomyxoma peritonei (PMP) is a rare abdominal cancer (incidence 2-3 people per million per year), characterized by accumulation of mucinous tumor tissue throughout the peritoneal cavity, but which rarely gives rise to distant metastases. The condition usually originates from ruptured mucinous tumors of the appendix and develops slowly, resulting in abdominal distention and compression of intra-abdominal organs. For histologically low-grade PMP, the prognosis is excellent with standard-of-care treatment involving cytoreductive surgery (CRS) and hyperthermic intraperitoneal chemotherapy (HIPEC), with a 10-year survival of up to 70%. However, for patients that cannot be cured by surgery, there are no good treatment options. Responses to systemic chemotherapy are generally poor, and biological agents, such as angiogenesis inhibitors, have been suggested as alternative therapeutic approaches, but so far with little success. In the setting of non-resectable and recurrent disease, PMP is therefore a debilitating and ultimately fatal condition, leaving patients to experience progressively poor quality of life caused by the increasing intraperitoneal tumor burden. As the tumor grows, extensive abdominal distention by mucinous tumor tissue will occur, resulting in compression of intraperitoneal organs and ultimately, death. Very little is known about immune cell infiltration and the tumor microenvironment in these tumors.

GNAS encodes for the alpha subunit of the heterotrimeric G-protein (Gαs). It is activated by the G-protein-coupled receptor and together they transmit signals from outside the cell to its interior through the cAMP signaling pathway (Bastepe, Horm Metab Res 2012 [22]). Gain-of-function mutations in GNAS, mainly Arg (R) 201 and to a lesser extent Gln (Q) 227, leads to constitutive activity and persistent signaling, and are found in various endocrine and nonendocrine tumors. How this molecular mechanism contributes to pathogenesis remains unclear and may be tissue specific. In pseudomyxoma peritonei (PMP), GNAS R201 is frequently mutated, with a median frequency of 44% across 13 studies, although the variation is quite large (17-100%).

There is a need to provide effective cancer vaccines and/or treatments where there are limited other treatment options. In particular, there is a need to provide vaccines and/or treatments which are targeted to PMP.

### SUMMARY OF INVENTION

GNAS represents a driver oncogene in a subset of highly prevalent cancers, such as colorectal (6%) and lung cancer (2%), and more frequently in rare tumor types like thyroid adenoma (23%), GH-secreting pituitary adenoma (41%), IPMN (58%), and intramuscular myxoma (45%) [8, 9]. A summary of the presence of GNAS R201 and Q227 mutations across different cancers can be seen in Table 1 which is adapted from Innamorati et al, 2008 [10].

TABLE 1
prevalence of GNAS R201 and Q227 mutations in different cancers

| | | Incidence |
|---|---|---|
| Site | Histology (Dysplasia/Neoplasia) | % (n) |
| Thyroid | Toxic thyroid adenoma | 23% (65) |
| | Non functional adenoma | 0% (31) |
| | Carcinoma | 0% (18) |
| Pituitary | GH-secreting adenoma | 41% (504) |
| | Prolactin-secreting adenoma | 0% (7) |
| | Non functioning | 3% (32) |
| Muscle | Intramuscolar myxoma | 45% (101) |
| | Various myxoid lesions | 0% (105) |
| Bone | Fibrous dysplasia (FD) | 81% (414) |
| | Low grade periosteal osteosarcoma | 0% (11) |
| | Low grade central osteosarcoma | 3% (35) |
| | Low grade parosteal osteosarcoma | 0% (80) |
| | Osteofibrous dysplasia | 0% (13) |
| | Ossifying fibroma | 0% (66) |
| Blood | Hematological conditions | 0.6% (512) |
| Kidney | Renal cell carcinoma | 17% (30) |
| Lung | Mucinous cystoadenoma | 0.5% (208) |
| Pancreas | Intraduct. Papill. Mucin, Neop. (IPMN) | 58% (809) |
| | Incipient IPMN | 33% (21) |
| | Intraduct. Tubulopapill. Neop. (ITPN) | 60% (15) |
| | Intraepithelial neoplasia (PanIN) | 2% (246) |
| | Serous cystoadenoma (SCN) | 0% (54) |
| | Mucin. Cyst. Neop (MCN) | 0% (31) |
| | Neuroendocrine tumor | 0% (52) |
| | Ductal adenocarcinoma (PDAC) | 0.4% (253) |
| Biliary tract | IPMN of the bile duct | 23% (120) |
| | Biliary intraepithelial neoplasia | 1% (82) |
| Stomach | Pyloric gland adenoma | 48% (23) |
| | Gastric adenocarc. Of the fundic gland | 24% (29) |
| | Hyperplastic gastric polyps | 0% (10) |
| | Foveolar type adenoma | 0% (23) |
| | Intestinal type adenoma | 0% (34) |
| | Gastric adenocarcinoma | 0% (71) |
| Duodenum | Pyloric gland adenoma | 92% (35) |
| | Gastric foveolar metaplasia | 41% (66) |
| | Gastric heterotopia | 28% (81) |
| | Adenocarcinoma | 17% (30) |
| | Gastroent. neuroen. tum. (GEP-NET) | 0% (31) |
| Colon-rectum | Villous adenoma | 67% (55) |
| | Tubular villous adenoma | 4% (154) |
| | Tubular adenoma | 0% (28) |
| | Polyps | 0% (45) |
| | Adenocarcinoma | 3% (820) |
| Liver | Normal liver Intrahepat. cholangioc | 12% (43) |
| | Advanc. Intrahepatic cholangiocarc | 3% (38) |
| | Hepatocell. Adenoma | 4% (179) |
| | Hepatocell. Carcinoma | 0.8% (245) |
| | Fluke-ass cholangiocarcinoma | 9.3% (53) |
| Appendix | Low grade app. muc. neop. (LAMN) | 43% (84) |
| | Adenocarcinoma | 46% (106) |
| Gonads | Leydig cell stromal tumor | 67% (6) |
| | Lobular Endocer. Glandular Hyperpl. | 28% (32) |
| | Juvenile Ovarian granul. cell tumor | 30% (30) |
| | Mucinous cystoadenoma | 9% (45) |
| | Mucinous border line tumor | 4% (53) |
| | Mucinous cystadenocarcinoma | 2% (45) |
| | Ovarian granulosa cell tumor | 0% (25) |
| | Other sex cord stromal tumors | 0% (6) |
| | Adenocarcinoma | 4% (92) |
| | Squamous cell carcinoma | 0% (43) |
| Adrenocortical | Cortisol producing adenoma | 20% (25) |
| | Adrenocrotical Carcinoma | 3% (40) |

As can be seen, the potential for exploiting this mutation in cancer therapy extends to a number of cancer entities.

Accordingly, in one aspect of the invention there is provided a peptide mixture suitable for eliciting an immune response, comprising a first peptide and a second peptide, wherein the first peptide comprises a first sequence of at least 8 amino acids, wherein said first sequence has at least 75% sequence identity to a first region of 8 amino acids of SEQ ID NO: 11 wherein said first region includes position 201 of the SEQ ID NO: 11 such that the first sequence includes the amino acid in position 201 of the SEQ ID NO: 11, and wherein the second peptide comprises a second sequence of at least 8 amino acids, wherein said second sequence has at least 75% sequence identity to a second region of 8 amino acids of SEQ ID NO: 12, and wherein said second region includes position 201 of the SEQ ID NO: 12 such that the second sequence includes the amino acid in position 201 of the SEQ ID NO: 12.

In some embodiments, each of the first and second peptides consists of no more than 100 amino acids, and preferably no more than 30 amino acids.

In some embodiments, the amino acid in position 201 of SEQ ID NO: 11 and/or 12 in the first sequence is flanked on each side by at least two, three, four or five amino acids, and the amino acid in position 201 of SEQ ID NO: 11 and/or 12 in the second sequence is flanked on each side by at least two, three, four or five amino acids.

In some embodiments, the sequence of 8 amino acids in the first peptide has 100% sequence identity to the first region of 8 amino acids including position 201 of SEQ ID NO: 11 and the second peptide has 100% sequence identity to the second region of 8 amino acids including position 201 of SEQ ID NO: 12.

In some embodiments, the first peptide comprises or consists of a sequence having at least 75% sequence identity to SEQ ID NOs: 1, 3, 5 or 7, preferably SEQ ID NO: 3, and the second peptide comprises a sequence having at least 75% sequence identity to SEQ ID NOs: 2, 4, 6 or 8, preferably SEQ ID NO: 4.

In another aspect of the invention, there is provided a peptide suitable for eliciting an immune response, wherein the peptide comprises a sequence of at least 8 amino acids, wherein the peptide consists of no more than 20 amino acids, and wherein said sequence has at least 75% sequence identity to a region of 8 amino acids of SEQ ID NO: 11, wherein said region includes position 201 of the SEQ ID NO: 11 such that said sequence includes the amino acid in position 201 of the SEQ ID NO: 11, or wherein said sequence has at least 75% sequence identity to a region of 8 amino acids of SEQ ID NO: 12, wherein said region includes position 201 of the SEQ ID NO: 12 such that said sequence includes the amino acid in position 201 of the SEQ ID NO: 12.

In some embodiments, the amino acid in position 201 of SEQ ID NO: 11 or 12 in the sequence is flanked on each side by at least two, three, four or five amino acids.

In some embodiments, the sequence of 8 amino acids in the peptide has 100% sequence identity to the region of 8 amino acids including position 201 of SEQ ID NO: 11 or SEQ ID NO:12.

In some embodiments, the peptide comprises or consists of a sequence having at least 75% sequence identity to any of SEQ ID NO: 1-8, preferably SEQ ID NOs: 3 or 4.

In some embodiments the peptide consists of a sequence having at least 75% sequence identity to SEQ ID NO: 1 or 2, the peptide consists of a sequence having at least 85% sequence identity to SEQ ID NO: 7 or 8, the peptide consists of a sequence consisting of 19 amino acids and having at least 95% sequence identity to SEQ ID NO: 5 or 6, or the peptide consists of a sequence consisting of 13 amino acids and having at least 90% sequence identity to SEQ ID NO: 3 or SEQ ID NO: 4.

In some embodiments, the peptide consists of a sequence having at least 80%, at least 85%, at least 90%, at least 95%, at least 97%, at least 98%, at least 99% or 100% sequence identity to SEQ ID NO: 1 or 2, the peptide consists of a sequence having at least 97%, at least 98%, at least 99% or 100% sequence identity to SEQ ID NO: 7 or 8, the peptide consists of a sequence consisting of 19 amino acids and having at least 97%, at least 98%, at least 99% or 100% sequence identity to SEQ ID NO: 5 or 6, or the peptide consists of a sequence consisting of 13 amino acids and having at least 95%, at least 97%, at least 98%, at least 99% or 100% sequence identity to SEQ ID NO:3 or SEQ ID NO:4.

In some embodiments, the peptide consists of the amino acid sequence of one of SEQ ID NOs: 1-8, preferably SEQ ID NO: 3 or SEQ ID NO: 4.

In some embodiments, the peptide is not identical to SEQ ID NO: 1 or 2. In some embodiments, the peptide is not identical to any of SEQ ID NOs: 1-8.

In yet another aspect of the invention, there is provided a T-cell mixture comprising T-cells specific for each of the peptides in one of the peptide mixtures as described above, or a T-cell preparation comprising a T-cell specific for a peptide as described above, when presented on an MHC molecule.

In another aspect of the invention, there is provided a nucleic acid molecule encoding a peptide as described above.

In a further aspect of the invention, there is provided at least one nucleic acid molecule, wherein the nucleic acid molecule or molecules individually or collectively comprise nucleotide sequences encoding at least two of the peptides from one of the peptide mixtures as described above, or at least one of the peptides as described above.

In another aspect of the invention, there is provided a vector comprising a nucleic acid molecule or at least one nucleic acid molecule as described above.

In yet another aspect of the invention, there is provided a host cell comprising the vector described above, preferably wherein said host cell is E. coli.

In a further aspect of the invention, there is provided a T cell receptor specific for a peptide as described above.

In another aspect of the invention, there is provided a pharmaceutical composition comprising a peptide mixture as described above, or a peptide according to the first or second peptide in a peptide mixture as described above, or a peptide as described above, or a T-cell mixture or preparation as described above, and a pharmaceutically acceptable carrier, diluent and/or excipient.

In some embodiments, the pharmaceutical composition further comprises at least one checkpoint inhibitor, preferably wherein the at least one checkpoint inhibitor comprises a PD-1 or PD-L1 inhibitor, and more preferably wherein the PD-1 or PD-L1 inhibitor is an anti-PD-1 or anti-PD-L1 antibody.

In one aspect of the invention, there is provided a peptide mixture as described above, or a peptide defined as the first or second peptide in any of the peptide mixtures described above, or a peptide as described above, or a T-cell mixture or preparation as described above, a nucleic acid molecule as described above, at least one nucleic acid molecule as described above or a pharmaceutical composition as described above, for use as a vaccine or a medicament.

In some embodiments, the use is for the prophylaxis and/or treatment of cancer.

In some embodiments, the use is by simultaneous, separate or sequential administration with at least one adjuvant, preferably GM-CSF.

In some embodiments, the use is by simultaneous, separate or sequential administration with at least one checkpoint inhibitor, preferably wherein the at least one checkpoint inhibitor comprises a PD-1 or PD-L1 inhibitor, and more preferably wherein the PD-1 or PD-L1 inhibitor is an anti-PD-1 or anti-PD-L1 antibody.

In another aspect of the invention, there is provided a method of treating cancer, comprising administering a peptide mixture described above, a peptide defined as the first or second peptide in any one of the peptide mixtures described above, a peptide as described above, a T-cell mixture or preparation as described above, a nucleic acid as described above, at least one nucleic acid molecule as described above or a pharmaceutical composition as described above, to a patient in need thereof.

In some embodiments, at least one adjuvant and/or one checkpoint inhibitor is also administered to the patient in need thereof, preferably wherein the at least one checkpoint inhibitor comprises a PD-1 or PD-L1 inhibitor, and more preferably wherein the PD-1 or PD-L1 inhibitor is an anti-PD-1 or anti-PD-L1 antibody.

In some embodiments of the use or method as described above, the cancer is pseudomyxoma peritonei (PMP).

In a further aspect of the invention, there is provided a peptide mixture, a peptide, a T-cell mixture, a T-cell preparation, a nucleic acid molecule, at least one nucleic acid molecule, or a pharmaceutical composition for use in a method comprising: i) identifying at least one GNAS mutation present in a sample taken from a patient; and ii) administering to a patient in need thereof having at least one GNAS mutation, a peptide mixture as described above, or a peptide defined as the first or second peptide in any one of peptide mixtures described above, a peptide as described above, a T-cell mixture or a T-cell preparation as described above, a nucleic acid molecule as described above, at least one nucleic acid molecule as described above or a pharmaceutical composition as described above.

### Definitions

In this specification, the following terms may be understood as follows:

The term “peptide” as used herein, refers to a polymer of amino acid residues that is (or has a sequence that corresponds to) a fragment of a longer protein. The term also applies to amino acid polymers in which one or more amino acid residues is a modified residue, or a non-naturally occurring residue, such as an artificial chemical mimetic of a corresponding naturally-occurring amino acid, as well as to naturally occurring amino acid polymers. The term “fragment”, as used herein, refers to a series of consecutive amino acids from a longer polypeptide or protein. In some embodiments, the peptide or the fragment is isolated, that is to say, the peptide or fragment is removed from the components in its natural environment.

The term “peptide mixture”, as used herein, refers to two or more peptides which are mixed but not chemically combined. The mixtures may be present as a dry powder, solution, suspension or colloid, and may be homogeneous or heterogeneous.

The term “amino acid” as used herein refers to naturally occurring and synthetic amino acids, as well as amino acid analogues and amino acid mimetics that have a function that is similar to naturally occurring amino acids. Naturally occurring amino acids are those encoded by the genetic code, as well as those modified after translation in cells (e.g. hydroxyproline, gamma-carboxyglutamate, and O-phosphoserine). The phrase “amino acid analogue” refers to compounds that have the same basic chemical structure (an alpha carbon bound to a hydrogen, a carboxy group, an amino group, and an R group) as a naturally occurring amino acid but have a modified R group or modified backbones (e.g. homoserine, norleucine, methionine sulfoxide, methionine methyl sulphonium). The phrase “amino acid mimetic” refers to chemical compounds that have different structures from, but similar functions to, naturally occurring amino acids. It is to be appreciated that, owing to the degeneracy of the genetic code, nucleic acid molecules encoding a particular polypeptide may have a range of polynucleotide sequences. For example, the codons GCA, GCC, GCG and GCT all encode the amino acid alanine.

The percentage “identity” between two sequences may be determined using the BLASTP algorithm version 2.2.2 (Altschul, Stephen F., Thomas L. Madden, Alejandro A. Schsffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402) using default parameters. In particular, the BLAST algorithm can be accessed on the internet using the URL http://www.ncbi.nlm.nih.gov/blast/.

The terms “gene”, “polynucleotides”, and “nucleic acid molecules” are used interchangeably herein to refer to a polymer of multiple nucleotides. The nucleic acid molecules may comprise naturally occurring nucleic acids (i.e. DNA or RNA) or may comprise artificial nucleic acids such as peptide nucleic acids, morpholin and locked nucleic acids as well as glycol nucleic acids and threose nucleic acids.

The term “nucleotide” as used herein refers to naturally occurring nucleotides and synthetic nucleotide analogues that are recognised by cellular enzymes.

The term “vector” as used herein refers to any natural or artificial construct containing a nucleic acid molecule in which the nucleic acid molecule can be subject to cellular transcription and/or translation enzymes. Exemplary vectors include: a plasmid, a virus (including bacteriophage), a cosmid, an artificial chromosome or a transposable element.

The term “host cell” as used herein refers to any biological cell which can be cultured in medium and used for the expression of a recombinant gene. Such host cells may be eukaryotic or prokaryotic and may be a microorganism such as a bacterial cell, or may be a cell from a cell line (such as an immortal mammalian cell line).

The term “conjugated” means reversibly combined or bound.

The term “immune response”, as used herein, refers in some embodiments to a T cell-mediated immune response upon presentation of a peptide by major histocompatibility (MHC) molecules on the surface of cells, and in particular refers to activation of T cells upon presentation of peptide.

The term “GNAS protein” as used herein, refers to the sequence shown in SEQ ID NO: 10. For the avoidance of doubt, the term “Gsα” may also be used interchangeably with “GNAS”.

The term “in vitro assay” means any experiment that is not carried out in a living organism. The person skilled in the art would be well aware of a variety of in vitro assays, but examples include: protein-based assays such as ELISAs, CBAs, ELISpots, immunoblotting, or nucleic acid based assays such as PCR, Northern or Southern blotting.

The terms “treatment” or “therapy” may be used interchangeably and refer to any partial or complete treatment and includes: inhibiting the disease or symptom, i.e. arresting its development; and relieving the disease or symptom, i.e. causing regression of the disease or symptom.

The term “medicament” means a composition suitable for treatment or therapy as defined above.

The term “pharmaceutical composition”, as used herein, means a pharmaceutical preparation suitable for administration to an intended human or animal subject for therapeutic purposes.

### BRIEF DESCRIPTION OF THE FIGURES

FIG. 1 shows that mutated GNAS peptides are immunogenic in PMP patients and healthy donors.

T-cell reactivity (shown by cellular division, proliferation) (SI, stimulation index) in blood samples from PMP patients is shown in (A). PBMC from 24 patients were pre-stimulated with mutated GNAS 30-mer peptides (R201C, SEQ ID NO: 1 and R201H, SEQ ID NO: 2). After 12-14 days, the T cells were re-stimulated with the mutated (SEQ ID NO: 1 or 2 as above) or WT GNAS 30-mer (SEQ ID NO: 9) peptides for 2 days before radioactive ³H-thymidine was added, which incorporates into the DNA of dividing cells. The following day, cells were harvested and the radioactivity measured. A Stimulation Index (SI) of ≥2 (above background) is considered as positive. SEC3 superantigen is included as a positive control of T-cell response. T cell proliferation in healthy donors for comparison is shown in (B). Paired, two-tailed t-tests were used to calculate the statistical significance of T-cell proliferation against GNAS WT peptide (SEQ ID NO: 9) versus other conditions. Individual responses for 24 of the PMP patients from (A) are shown in (B).

FIG. 2 shows that activation and exhaustion markers are upregulated on tumour infiltrating T cells in PMP patients.

Immunoprofiling of all patients with lymphocyte subpopulations by mass cytometry (CyTOF) is visualised on CD4 and CD8 T cells for 18 PMP patients, Shows the ratio of CD4:CD8 T cells in a subset of patients (A) and % of T cells expressing immune checkpoint molecules TIM-3, TIGIT, PD-1, LAG-3 and CXCR4 (B).

FIG. 3 shows that longer mutant GNAS peptides (≥25mers) are more immunogenic in healthy donors than shorter peptides (13-19mers) carrying the same point mutations. T-cell reactivity (shown by cellular division, proliferation) (SI, stimulation index) in blood samples from healthy donors is shown, reactivity to peptides with a R201C mutation are shown in (A), and reactivity to peptides with a R201H mutation are shown in (B). PBMC from 5 healthy donors (HD1-5) were pre-stimulated with mutated GNAS 25-mer peptides (R201C-25, SEQ ID NO: 7 and R201H-25, SEQ ID NO: 8) for approximately 14 days. The PBMCs were then re-stimulated with mutated GNAS peptides of varying lengths (13, 19, 25, 30 amino acids long) or controls (SEC-3 or WT (SEQ ID NO: 9) for 2 days before radioactive ³H-thymidine was added, which incorporates into the DNA of dividing cells. The following day, cells were harvested and the radioactivity measured. A Stimulation Index (SI) of ≥2 (above background) is considered as positive. SEC3 superantigen is included as a positive control of T-cell response. In (A) the following mutant peptides were used for re-stimulation: R201C-30 SEQ ID NO: 1; R201C-25—SEQ ID NO: 7; R201C-19—SEQ ID NO: 5; R201C-13—-SEQ ID NO: 3. In (B) the following mutant peptides were used for re-stimulation: R201H-30—SEQ ID NO: 2; R201H-25—SEQ ID NO: 8; R201H-19—SEQ ID NO: 6; R201H-13—SEQ ID NO: 4. HD=healthy donor. The dotted line at SI≥2 defines the cut-off for a positive response.

FIG. 4 shows that that GNAS is an ideal cancer antigen to target.

Adapted from Hollingsworth & Jansen, 2019 [7].

FIG. 5 shows the proposed study flow chart for a signal-finding, prospective, open-label, phase I trial for a cancer vaccine targeting GNAS combined with immunotherapy for patients with PMP.

FIG. 6 shows the therapeutic concept. Mutated Gsα peptides act as neo-antigens and elicit a spontaneous, anti-tumor specific T cell response in PMP patients, but this is not sufficient to control tumor growth. Without wishing to be bound by theory, it is believed that vaccination with mutated Gsα peptides will amplify the existing response and induce de novo responses of naïve T cells, resulting in a clonal expansion of T cells recognizing mutated Gsα. The anti-tumor immune response can then be boosted by adding an immune checkpoint inhibitor (ICI) to remove the inhibition caused by up-regulation of immune checkpoint molecules on tumor-infiltrating T cells, restoring a functional immune response.

FIG. 7 shows the change in the CD4/CD8 ratio after stimulation of PBMCs with short peptides (SEQ ID NOs: 13-15) and long peptides (SEQ ID NOs: 1 and 2). Simulation of PBMCs with short peptides leads to a lower CD4/CD8 ratio than stimulation with the long peptides. Stimulation with shorter peptides therefore generates more CD8 T cells than CD4 T cells, and longer peptides stimulate more CD4 T cells than CD8 cells.

FIG. 8 shows T-cell proliferation after stimulation with short peptides (SEQ ID NOs: 13-15) and long peptides (SEQ ID NOs: 1 and 2). Long mutant GNAS peptides (SEQ ID NOs: 1 and 2) are more immunogenic in healthy donors than short peptides (SEQ ID NOs: 13-15) carrying the same point mutations. Stimulation with short peptides induces lower proliferation compared to background without peptide versus stimulation with long peptides in the donor shown.

### DETAILED DESCRIPTION OF THE INVENTION

In Norway, curatively aimed treatment for PMP is offered only at the Norwegian Radium Hospital, OUS at the National Unit for treatment of peritoneal surface malignancies, and 10-15 cases are treated every year. An extensive translational research program headed by Professor Kjersti Flatmark has been developed with the aim of understanding this rare disease and developing new treatment strategies. Tumor tissue and blood samples have been collected from patients with PMP since 2009, and this database of treated PMP patients comprises more than 200 cases.

The main mutational drivers in PMP (present in 70-80% of tumors) are distinct activating mutations in the KRAS and GNAS oncogenes [1, 2]. The GNAS mutations (R201H and R201C) are of particular interest, since these are extremely rare in other cancers, and seem to be linked to mucin production in PMP.

As such, the present invention provides a peptide mixture suitable for eliciting an immune response, comprising a first peptide and a second peptide, wherein the first peptide comprises a first sequence of at least 8 amino acids, wherein said first sequence has at least 75% sequence identity to a first region of 8 amino acids of SEQ ID NO: 11, wherein said first region includes position 201 of SEQ ID NO: 11 such that the first sequence includes the amino acid in position 201 of SEQ ID NO: 11, and wherein the second peptide comprises a second sequence of at least 8 amino acids, wherein said second sequence has at least 75% sequence identity to a second region of x amino acids of SEQ ID NO: 12, and wherein said second region includes position 201 of SEQ ID NO: 12 such that the second sequence includes the amino acid in position 201 of the SEQ ID NO: 12. This peptide mixture provides prophylaxis and/or treatment of cancer which is cost effective, and without needing to identify the specific GNAS mutation present in the cancer. In particular, Table 2 shows that all GNAS mutations detected were R201H or R201C mutations.

The skilled person will also understand that, in some embodiments, the first region includes position 200, 201 and 202 of the SEQ ID NO: 11 and thus the first sequence includes the three consecutive amino acids in positions 200, 201 and 202 of the SEQ ID NO: 11, and the second region includes positions 200, 201 and 202 of the SEQ ID NO: 12 and thus the second sequence includes the three consecutive amino acids in positions 200, 201 and 202 of the SEQ ID NO: 12.

The term “position 201 of SEQ ID NO: 11”, as used herein, means an amino acid in the peptide of SEQ ID NO: 11 located in the peptide's amino acid chain at a position corresponding to the 201^{st} amino acid, counting from the N-terminal. Corresponding meanings are attributed to the amino acid equivalent to position 200 and 202 of SEQ ID NO: 11. Accordingly, the term “position 201 of SEQ ID NO: 12”, as used herein, means an amino acid in a peptide of SEQ ID NO: 12 located in the peptide's amino acid chain at a position corresponding to the 201^{st} amino acid counting from the N-terminal. Again, corresponding meanings are attributed to the amino acid equivalent to 200 and 202 of SEQ ID NO: 12.

In some embodiments, the peptide mixture is suitable for eliciting an immune response against GNAS, preferably mutated GNAS, and more preferably R201C and R201H mutated GNAS.

In some embodiments, the first sequence of at least 8 amino acids, wherein said first sequence has at least 80%, 85%, 90%, 95%, 99% or 100% sequence identity to a first region of 8 amino acids of SEQ ID NO: 11, and/or the second sequence of at least 8 amino acids, wherein said first sequence has at least 80%, 85%, 90%, 95%, 99% or 100% sequence identity to a first region of 8 amino acids of SEQ ID NO: 12.

Thus in some embodiments, one or more amino acids of the peptides are omitted or are substituted for a different amino acid, preferably a similar amino acid. A similar amino acid is one which has a side chain moiety with related properties and the naturally occurring amino acids may be categorized into the following groups. The group having basic side chains: lysine, arginine, histidine. The group having acidic side chains: aspartic acid and glutamic acid. The group having uncharged polar side chains: aspargine, glutamine, serine, threonine and tyrosine. The group having non-polar side chains: glycine, alanine, valine, leucine, isoleucine, proline, phenylalanine, methionine, tryptophan and cysteine. Therefore, it is preferred to substitute amino acids within these groups and the substitution of a “similar” amino acid residue is a substitution within one of the aforementioned groups (this is also known as a “conservative substitution”).

In some embodiments, each of the first and second peptides consists of no more than 100 amino acids. Preferably, each of the first and second peptides consists of no more than 30 amino acids. In some embodiments, each of the first and second peptides consists of 13 or 19 amino acids. Preferably, each of the first and second peptides consists of 30 amino acids.

In some embodiments, the amino acid in position 201 of SEQ ID NO: 11 and/or 12 in the first sequence is flanked on each side by at least two, three, four or five amino acids, and the amino acid in position 201 of SEQ ID NO: 11 and/or 12 in the second sequence is flanked on each side by at least two, three, four or five amino acids.

In certain embodiments, the sequence of 8 amino acids in the first peptide has 100% sequence identity to the first region of 8 amino acids including position 201 of SEQ ID NO: 11 and the second peptide has 100% sequence identity to the second region of 8 amino acids including position 201 of SEQ ID NO: 12.

In some embodiments, the first peptide comprises a sequence having at least 75% sequence identity to SEQ ID NOs: 1, 3, 5 or 7 (preferably SEQ ID NO: 3) and the second peptide comprises a sequence having at least 75% sequence identity to SEQ ID NOs: 2, 4, 6 or 8. (preferably SEQ ID NO: 4). The skilled person will understand that “comprises” may also mean “consists of”, and accordingly, in some embodiments, the first peptide consists of a sequence having at least 75% sequence identity to SEQ ID NOs: 1, 3 5 or 7 and the second peptide consists of a sequence having at least 75% sequence identity to SEQ ID NOs: 2, 4, 6 or 8. In preferred embodiments, the first peptide consists of a sequence having at least 75% sequence identity to SEQ ID NO: 3 and the second peptide consists a sequence having at least 75% sequence identity to SEQ ID NO: 4. In some embodiments, the first peptide consists of a sequence having at least 75% sequence identity to SEQ ID NO: 1 and the second peptide consists of a sequence having at least 75% sequence identity to SEQ ID NO: 2. In some embodiments, the sequence identity is at least 80%, 85%, 90%, 95%, 97%, 98%, or 99%, or is 100%.

In some embodiments, the first peptide has at least 75% sequence identity to SEQ ID NO: 1, 3, 5 or 7 and the second peptide has at least 75% sequence identity to SEQ ID NO: 2, 4, 6 or 8. In some embodiments, the first peptide has at least 80%, 85%, 90%, 95%, 97%, 98%, or 99% sequence identity, or is 100% identical, to SEQ ID NO: 1, 3, 5 or 7, and the second peptide has at least 80%, 85%, 90%, 95%, 97%, 98%, or 99% sequence identity, or is 100% identical, to SEQ ID NO: 2, 4, 6 or 8.

In some embodiments, each of the first and second peptide comprises at least 19 amino acids.

In some embodiments, the first sequence of the first peptide is not identical to SEQ ID NO: 1 and the second sequence of the second peptide is not identical to SEQ ID NO: 2. In some embodiments, the first peptide is not identical to SEQ ID NO: 1 and the second peptide is not identical to SEQ ID NO: 2. In some embodiments, none of the peptides is identical to any of SEQ ID NOs: 1-8.

In some embodiments, the first sequence of the first peptide comprises at least 19 amino acids and the second sequence of the second peptide comprises at least 19 amino acids, wherein the first peptide is not identical to SEQ ID NO: 1 and the second peptide is not identical to SEQ ID NO: 2. In some embodiments, the first peptide comprises at least 19 amino acids and is not identical to SEQ ID NO: 1 and the second peptide comprises at least 19 amino acids and is not identical to SEQ ID NO: 2. In some embodiments, each of the peptides comprises at least 19 amino acids and none of the peptides is identical to SEQ ID NO: 1 or 2, preferably none of the peptides is identical to any of SEQ ID NOs: 1-8.

In some embodiments, the first peptide consists of the sequence of SEQ ID NO: 1 and the second peptide consists of the sequence of SEQ ID NO: 2. In particular, FIGS. 7 and 8 show that this peptide mixture increases the CD4/CD8 T-cell ratio, as well as T-cell proliferation, more than a mixture of previously known peptides. Thus, this peptide mixture of the invention is expected to provide more effective treatment and/or prophylaxis of cancer than previously known peptides of mutated GNAS.

The peptide mixtures of the present invention may contain the peptides in equal or in different proportions. In some embodiments, the first and second peptides are present in the mixture in equal proportions, i.e. each peptide comprises 50% of the peptide component of the peptide mixture. In other embodiments, there is a greater proportion of the first peptide in the peptide mixture than the second peptide. For example, the first peptide may comprise at least 55%, at least 60%, at least 70%, at least 80% or at least 90% of the peptide component of the peptide mixture. In alternative embodiments, there is a greater proportion of the second peptide in the peptide mixture than the first peptide. For example, the second peptide may comprise at least 55%, at least 60%, at least 70%, at least 80% or at least 90% of the peptide component of the peptide mixture.

The present invention also provides a peptide suitable for eliciting an immune response, wherein the peptide comprises a sequence of at least 8 amino acids, wherein the peptide consists of no more than 20 amino acids, and wherein said sequence has at least 75% sequence identity to a region of 8 amino acids of SEQ ID NO: 11, wherein said region includes position 201 of the SEQ ID NO: 11 such that said sequence includes the amino acid in position 201 of the SEQ ID NO: 11, or wherein said sequence has at least 75% sequence identity to a region of 8 amino acids of SEQ ID NO: 12, wherein said region includes position 201 of the SEQ ID NO: 12 such that said sequence includes the amino acid in position 201 of the SEQ ID NO: 12.

In some embodiments, the peptide comprises at least 19, at least 20, at least 21, at least 22, at least 23, at least 24, at least 25, at least 26, at least 27, at least 28, at least 29 or at least 30 amino acids.

In some embodiments, the peptide comprises no more than 30, no more than 29, no more than 28, no more than 27, no more than 26, no more than 25, no more than 24, no more than 23, no more than 22, no more than 21, no more than 20 or no more than 19 amino acids.

In some embodiments, the peptide consists of 13 or 19 amino acids. In some embodiments, the peptide consists of 25 or 30 amino acids.

In some embodiments, the amino acid in position 201 of SEQ ID NO: 11 or 12 in the sequence is flanked on each side by at least two, three, four or five amino acids.

In some embodiments, wherein the sequence of 8 amino acids in the peptide has 100% sequence identity to the region of 8 amino acids including position 201 of SEQ ID NO: 11 or SEQ ID NO: 12.

In some embodiments, wherein the peptide comprises or consists of a sequence having at least 75% sequence identity to any of SEQ ID NOs: 1-8. Preferably, the peptide consists of a sequence having at least 75% sequence identity to SEQ ID NOs: 3 or 4. In some embodiments, the sequence identity is at least 80%, 85%, 90%, 95%, 97%, 98%, or 99%, or is 100%.

In some embodiments, the peptide consists of a sequence having at least 75%, 80%, 85%, 90%, 95%, 97%, 98%, or 99%, or is 100% to one of SEQ ID NOs: 1-8. In some embodiments, the peptide consists of a sequence having at least 75% sequence identity to SEQ ID NO: 1 or 2, the peptide consists of a sequence having at least 85% sequence identity to SEQ ID NO: 7 or 8, the peptide consists of a sequence consisting of 19 amino acids and having at least 95% sequence identity to SEQ ID NO: 5 or 6, or the peptide consists of a sequence consisting of 13 amino acids and having at least 90% sequence identity to SEQ ID NO: 3 or SEQ ID NO: 4. In some embodiments, the peptide consists of a sequence having at least 80%, at least 85%, at least 90%, at least 95%, at least 97%, at least 98%, at least 99% or 100% sequence identity to SEQ ID NO: 1 or 2, the peptide consists of a sequence having at least 97%, at least 98%, at least 99% or 100% sequence identity to SEQ ID NO: 7 or 8, the peptide consists of a sequence consisting of 19 amino acids and having at least 97%, at least 98%, at least 99% or 100% sequence identity to SEQ ID NO: 5 or 6, or the peptide consists of a sequence consisting of 13 amino acids and having at least 95%, at least 97%, at least 98%, at least 99% or 100% sequence identity to SEQ ID NO: 3 or SEQ ID NO: 4. Preferably, the peptide consists of the amino acid sequence of SEQ ID NO: 3 or SEQ ID NO: 4.

In some embodiments, the sequence identity is calculated relative to the reference peptide. Thus, for example, when the peptide of the invention has at least 75% sequence identity to SEQ ID NO: 1 or SEQ ID NO: 2, this means that the peptide of the invention comprises at least 75% of the amino acids of SEQ ID NO: 1 or SEQ ID NO: 2. For example, when the peptide has 75% sequence identity to SEQ ID NO: 1 or SEQ ID NO: 2, the peptide can be 23 amino acids long and be identical to a corresponding sequence of 23 amino acids in SEQ ID NO: 1 or SEQ ID NO: 2 (which have 30 amino acids; 75% of 30 amino acids is 22.5 amino acids), or the peptide can longer than 23 amino acids and has 23 amino acids which are identical to corresponding amino acids in SEQ ID NO: 1 or SEQ ID NO: 2. The same principle applies when the peptide has at least 80%, 85%, 90%, 95%, 97%, 98%, 99% or 100% sequence identity to the reference sequence. Thus, in some embodiments, the peptide comprises at least 75%, 80%, 85%, 90%, 95%, 97%, 98%, 99% or 100% of the amino acids of one of SEQ ID NOs: 1-8, preferably of the amino acids of SEQ ID NO: 3 or SEQ ID NO: 4. This can be calculated using the EMBOSS Needle algorithm at https://www.ebi.ac.uk/Tools/psa/emboss needle/.

In some embodiments, the peptide or a peptide in a peptide mixture as described above, the peptide may comprise non-peptide elements, for example, if it is conjugated to a non-peptide moiety.

The present invention also provides a T-cell mixture comprising T-cells specific for each of the peptides in one of the peptide mixtures as described above, or a T-cell preparation comprising a T-cell specific for a peptide as described above, when presented on an MHC molecule.

There is also provided a nucleic acid molecule encoding a peptide described above. There is further provided a nucleic acid mixture comprising a first nucleic acid molecule and a second nucleic acid molecule, wherein the first nucleic acid molecule encodes a first peptide as described above, and the second nucleic acid molecule encodes a second peptide a described above. There is further provided at least one nucleic acid molecule wherein the nucleic acid molecule or molecules individually or collectively comprise nucleotide sequences encoding at least two of the peptides in a peptide mixture as described above or at least one of the peptides as described above.

The present invention also provides a vector comprising a nucleic acid molecule or a nucleic acid mixture as described above. The present invention also provides a vector comprising a nucleic acid molecule or at least one nucleic acid molecule as described above. There is also provided a host cell comprising the described vector. Preferably, the host cell is E. coli.

In addition to the sequence specifically encoding the protein of the invention, the nucleic acid molecule or the at least one nucleic acid molecule may contain other sequences such as primer sites, transcription factor binding sites, vector insertion sites and sequences which resist nucleolytic degradation (e.g. polyadenosine tails). The nucleic acid molecule or the at least one nucleic acid molecule may be DNA or RNA and may include synthetic nucleotides, provided that the polynucleotide is still capable of being translated in order to synthesize a protein of the invention. In another aspect, there is a vector comprising such a nucleic acid sequence; in a further aspect, there is a host cell comprising said vector, preferably wherein the host cell is E. coli.

In addition to the nucleic acid molecule and at least one nucleic acid molecule described above, the plasmid comprises other elements such as a prokaryotic origin of replication (for example, the E. coli OR1 origin of replication) an autonomous replication sequence, a centromere sequence; a promoter sequence, upstream of the nucleic acid sequence, a terminator sequence located downstream of the nucleic acid sequence, an antibiotic resistance gene and/or a secretion signal sequence. A vector comprising an autonomous replication sequence is also a yeast artificial chromosome. In some alternative embodiments, the vector is a virus, such as a bacteriophage and comprises, in addition to the nucleic acid molecule or at least one nucleic acid molecule of the invention, nucleic acid sequences for replication of the bacteriophage, such as structural proteins, promoters, transcription activators and the like.

The nucleic acid molecule or at least one nucleic acid molecule of the invention may be used to transfect or transform host cells in order to synthesize the protein of the invention. Suitable host cells include prokaryotic cells such as E. coli and eukaryotic cells such as yeast cells, or mammalian or plant cell lines. Host cells are transfected or transformed using techniques known in the art such as electroporation; calcium phosphate base methods; a biolistic technique or by use of a viral vector.

After transfection, the nucleic acid molecule or at least one nucleic acid molecule of the invention is transcribed as necessary and translated. In some embodiments, the synthesized protein is allowed to remain in the host cell and cultures of the recombinant host cell are subsequently used. In other embodiments, the synthesized protein is extracted from the host cell, either by virtue of its being secreted from the cell due to, for example, the presence of secretion signal in the vector, or by lysis of the host cell and purification of the protein therefrom.

The present invention also provides a T cell receptor specific for a peptide as described above. The T cell receptor may be a α:β or a γ:δ T cell receptor. In some embodiments, said T cell receptor is bound to a peptide as described above when presented on an MHC molecule. However, in some embodiments, said T cell receptor is bound to a peptide as described above not presented on an MHC molecule, as it has been found that some types of T cell receptor can bind antigen alone (i.e. not displayed on an MHC molecule).

There is further provided a pharmaceutical composition comprising a peptide mixture, a peptide, or a T-cell mixture or preparation as described above, and a pharmaceutically acceptable carrier, diluent and/or excipient. In some embodiments, the at least one pharmaceutically acceptable carrier, diluent or excipient is physiological saline, phosphate buffered saline (PBS) and/or sterile water. In some embodiments, the pharmaceutical composition consists essentially of the peptide of the invention.

In some embodiments, the pharmaceutical composition further comprises at least one checkpoint inhibitor.

The term “immune checkpoint” as used herein refers to any point at which an immune response is limited. Immune checkpoints are inhibitory pathways that slow down or stop immune reactions and prevent excessive tissue damage from uncontrolled activity of immune cells. Examples of an “immune checkpoint” include the programmed cell death protein 1 (PD-1) checkpoint.

In the present invention, an immune checkpoint inhibitor is any compound, substance or composition (e.g. any small molecule chemical compound, antibody, nucleic acid molecule, or polypeptide, or fragments thereof) that is capable of down-regulating or blocking an immune checkpoint to allow more extensive immune activity. A checkpoint inhibitor may target PD-1, PD-L1 or TIGIT. It is preferred that the immune checkpoint inhibitor targets the PD-1 checkpoint or PD-L1 which is an endogenous ligand of PD-1.

In some embodiments, targeting an immune checkpoint is accomplished with an inhibitory antibody, or antigen-binding fragment thereof or a similar molecule. In a preferred embodiment, the immune checkpoint inhibitor is an antibody that specifically binds to a protein involved in the immune checkpoint pathway thereby disrupting and down-regulating the overall activity of the immune checkpoint. It is particularly preferred that the immune checkpoint inhibitor is an anti-PD-1 or an anti-PD-L1 antibody. It is especially preferred that the anti-PD-1 antibody is nivolumab or pembrolizumab, or the anti-PD-L1 antibody is avelumab.

In some embodiments, the immune checkpoint inhibitor is a small molecule antagonist that interferes with and/or inhibits the activity of a protein involved in the immune checkpoint pathway and thereby down-regulates the overall activity of the immune checkpoint. In a preferred embodiment, the small molecule antagonist targets the PD-1 proteins in order to down-regulate the PD-1 checkpoint (i.e. the small molecule antagonist is a small molecule PD-1 antagonist).

In some embodiments, there is a use of a peptide, or a nucleic acid molecule as described herein, in an in vitro assay. The person skilled in the art is well aware of a variety of in vitro assays, but examples include protein-based assays such as ELISAs, CBAs, ELISpots, immunoblotting, or nucleic acid based assays such as PCR, northern or southern blotting.

The present invention also provides a peptide mixture, a peptide, a T-cell mixture or preparation, a nucleic acid molecule, at least one nucleic acid molecule, a nucleic acid mixture, or a pharmaceutical composition as described above, for use as a vaccine or a medicament. Such a medicament may be administered to a patient in need thereof in any way known to the person skilled in the art. For example, among other techniques, the peptide mixture, peptide or pharmaceutical composition may be administered to a subject by injection, in the form of a solution, in the form of liposomes or in dry form (for example, in the form of coated particles, etc). In some embodiments, the peptide mixture, peptide or pharmaceutical composition may be administered in an amount, for example, of between 1 μg and 1 g of each peptide once every three days, once a week, once a month, once every three months, once every four months or once every six months. In one embodiment, the peptide mixture, peptide or pharmaceutical composition is administered on days 1, 8, 15 and 22, with further administration at six week intervals, preferably five further administrations e.g. at weeks 12, 18, 24, 30, 42 and 48. In one embodiment, the peptide mixture, peptide or pharmaceutical composition is delivered at a dose of 0.01 to 10 mg per administration. The skilled person will understand that this represents a suitable dose to obtain a technical effect in an individual to be treated.

In some embodiments, the vaccine or medicament comprising a peptide mixture, a peptide, a T-cell mixture or preparation, a nucleic acid molecule, at least one nucleic acid molecule, a nucleic acid mixture, or a pharmaceutical composition as described above, is administered after tumor debulking or elimination of the tumor by surgery.

The T-cell mixtures and T-cell preparations of the present invention may be administered by intra-venous injection and/or infusion, and may be administered in an amount, for example, of between 10⁶ and 10¹² of each T-cell specific for a peptide of the peptide mixture or peptide once every month, once every two months, once every three months, once every six months or once a year. Preferably, the dosage is administered once every month for between 2 and 5 months and is subsequently administered less frequently.

In some embodiments, the use is for the prophylaxis and/or treatment of cancer. In particular, cancers associated with mutations in a GNAS gene/protein, such as those tabulated in FIG. 5. Cancers may include adrenal gland, autonomic ganglia, biliary tract, bone, breast, central nervous system, cervical, colorectal, endometrial, haematopoietic, lymphoid, kidney, large intestine, liver, lung, oesophagus, ovarian, pancreatic, prostate, salivary gland, skin, small intestine, stomach, testicular, thymus, thyroid, upper aerodigestive tract and urinary tract cancer, and malignant melanoma and the peptide mixtures, peptides, T-cell mixtures and T-cell preparations of the present invention may be used for the prophylaxis and/or treatment of more than one of these types of cancer. Preferably, the cancer is pseudomyxoma peritonei (PMP).

In some embodiments, the use is by simultaneous, separate or sequential administration with at least one adjuvant, preferably GM-CSF. That is to say, the peptide mixture peptide, T-cell mixture or preparation, nucleic acid molecule, at least one nucleic acid molecule, nucleic acid mixture or pharmaceutical composition of the invention; and the adjuvant; (which may be described as “agents”) may be administered at a different time, as well as in a substantially simultaneous manner. The term simultaneously as used herein refers to administration of one or more agents at the same time. For example, in certain embodiments, the peptide or peptide mixture of the invention and the immune checkpoint inhibitor are administered simultaneously. Simultaneously includes administration contemporaneously, that is during the same period of time. In certain embodiments, the one or more agents are administered simultaneously in the same hour, or simultaneously in the same day. In some embodiments, the term “sequentially” refers to the one or more agents being administered within 1, 3, 5, 7, 10, 30 or 60 days of each other. In some embodiments, the term “sequentially” refers to the one or more agents being administered within 2, 4 or 6 months of each other.

Preferably, the use is by separate administration with at least one adjuvant. More preferably, the use is after separate administration with the at least one adjuvant.

An adjuvant is a compound that, when administered in conjunction with an antigen (e.g. the peptide or peptide mixture of the invention), effectively potentiate the host antigen-specific immune responses compared to responses raised when antigen is given alone. Possible adjuvants include analgesic adjuvants, inorganic compounds (such as alum, aluminium hydroxide, aluminium phosphate, calcium phosphate hydroxide), mineral oil (such as paraffin oil), bacterial product (such as killed bacteria Bordetella pertussis, Mycobacterium bovis, toxoids), non-bacterial organics (such as squalene), delivery systems (such as detergents like Quil A), plant saponins, cytokine (such as IL-1, IL-2, IL-12), combinations (such as Freund's complete adjuvant or Freund's incomplete adjuvant), or food-based oils. Preferably, the adjuvant is GM-CSF.

In another embodiment, the peptide mixture, peptide, T-cell mixture or preparation, nucleic acid molecule, at least one nucleic acid molecule, nucleic acid mixture or pharmaceutical composition for use described above is for use by simultaneous, separate or sequential administration with at least one checkpoint inhibitor. In some embodiments, the at least one checkpoint inhibitor comprises a PD-1 or PD-L1 inhibitor. In specific embodiments, the PD-1 or PD-L1 inhibitor is an anti-PD-1 or anti-PD-L1 antibody. In certain embodiments, the anti-PD-1 antibody is nivolumab or pembrolizumab, or the anti-PD-L1 antibody is avelumab.

There is also provided a method of treating cancer, comprising administering a peptide mixture, a peptide, a T-cell mixture or preparation, a nucleic acid molecule, at least one nucleic acid molecule, a nucleic acid mixture or a pharmaceutical composition as described above, to a patient in need thereof.

In some embodiments, the method comprises administering at least one adjuvant, preferably GM-CSF as above, by simultaneous, separate or sequential administration as defined above.

In some embodiments, the method further comprises administering at least one checkpoint inhibitor to the patient in need thereof. As above, possible checkpoint inhibitors include PD-1 inhibitors and PD-L1 inhibitors, TIGIT inhibitors. Preferably, the checkpoint inhibitor is a PD-L1 or PD-1 inhibitor. In preferred embodiments, the PD-1 or PD-L1 inhibitor is an anti-PD-1 or anti-PD-L1 antibody. In certain embodiments, the anti-PD-1 antibody is nivolumab or pembrolizumab, or the anti-PD-L1 antibody is avelumab.

In some embodiments, the peptide mixture, peptide, T-cell mixture or preparation, nucleic acid molecule, at least one nucleic acid molecule, nucleic acid mixture or pharmaceutical composition is administered on multiple occasions. In some embodiments, the peptide mixture peptide, T-cell mixture or preparation, nucleic acid molecule, at least one nucleic acid molecule, nucleic acid mixture or pharmaceutical composition is administered on at least three occasions.

Conveniently, the peptide mixture peptide, T-cell mixture or preparation, nucleic acid molecule, at least one nucleic acid molecule, nucleic acid mixture or pharmaceutical composition is administered every 5 days, weekly, fortnightly or monthly for a period of one to six months. Ideally, the peptide mixture peptide, T-cell mixture or preparation, nucleic acid molecule, at least one nucleic acid molecule, nucleic acid mixture or pharmaceutical composition is administered on days 1, 8, 15, 22.

Preferably, the peptide mixture peptide, T-cell mixture or preparation, nucleic acid molecule, at least one nucleic acid molecule, nucleic acid mixture or pharmaceutical composition is further administered at 3 to 9 week intervals thereafter. Still preferably, the peptide mixture peptide, T-cell mixture or preparation, nucleic acid molecule, at least one nucleic acid molecule, nucleic acid mixture or pharmaceutical composition is further administered at 6 week intervals thereafter (i.e. weeks 12, 18, 24, 30, 42 and 48). These further administrations may be referred to as “boost” administrations.

In some embodiments, an immune checkpoint inhibitor is administered after the peptide mixture peptide, T-cell mixture or preparation, nucleic acid molecule, at least one nucleic acid molecule, nucleic acid mixture or pharmaceutical composition is administered. Alternatively, an immune checkpoint inhibitor is administered on the same day or at the same time that the peptide mixture peptide, T-cell mixture or preparation, nucleic acid molecule, at least one nucleic acid molecule, nucleic acid mixture or pharmaceutical composition is administered. Alternatively still, an immune checkpoint inhibitor is administered before the peptide mixture peptide, T-cell mixture or preparation, nucleic acid molecule, at least one nucleic acid molecule, nucleic acid mixture or pharmaceutical composition is administered.

Preferably, an immune checkpoint inhibitor is administered between 6 weeks and 18 weeks after the peptide mixture peptide, T-cell mixture or preparation, nucleic acid molecule, at least one nucleic acid molecule, nucleic acid mixture or pharmaceutical composition is first administered. Ideally, an immune checkpoint inhibitor is then administered at the schedule recommended in the art for that particular checkpoint inhibitor, for example every 2 weeks for a total treatment period of 1 year.

In some embodiments, the use or treatment method for cancer is for pseudomyxoma peritonei (PMP).

In some embodiments, the use or treatment method is carried out after tumor debulking or elimination of the tumor by surgery.

There is further provided a peptide mixture, a peptide, a T-cell mixture, a T-cell preparation, a nucleic acid mixture, nucleic acid molecule, at least one nucleic acid molecule or a pharmaceutical composition for use in a method comprising:
i) identifying at least one GNAS mutation present in a sample taken from a patient; and
ii) administering to a patient in need thereof having at least one GNAS mutation, a peptide mixture, a peptide, a T-cell mixture or a T-cell preparation, a nucleic acid molecule, at least one nucleic acid molecule, a nucleic acid mixture or a pharmaceutical composition as described above. Preferably, the peptide mixture, peptide, T-cell mixture, T-cell preparation, nucleic acid molecule, at least one nucleic acid molecule, nucleic acid mixture or pharmaceutical composition is administered to the patient when the GNAS mutation identified in step i) is a R201H or a R201C mutation.

### EXAMPLES

### Methodology

### Clinical Samples

The study was approved by the Regional Ethics Committee of South-Eastern Norway (Project ID #10622 and #30634), and written informed consent was required for participation. Patients with suspected PMP were included in the study between April 2018 and May 2020 when admitted for CRS-HIPEC at the Norwegian Radium Hospital, Oslo University Hospital Comprehensive Cancer Center. Tumor and peripheral blood samples were collected at the time of surgery. Tumor distribution on the peritoneal surface was classified by the surgeon according to the peritoneal cancer index (PCI), giving a score between 0 and 39 [14]. Residual tumor after CRS was classified using the completeness of cytoreduction (CC) score (CC-0, no residual tumor; CC-1, residual tumor <0.25 cm; CC-2, tumor between 0.25 cm-2.5 cm and CC-3, tumor >2.5 cm) [14]. Complete cytoreduction was defined as CC-0 and CC-1. All PMP cases were evaluated by an expert pathologist and classified according to the Peritoneal Surface Oncology Group International (PSOGI) classification. Peripheral blood was collected from anonymous healthy donors for testing of immune responses upon informed consent (Project ID #2019/121).

Twenty-eight PMP patients were included in the study, 19 women and 9 men (Table 2). Of these, 27 had appendiceal primary tumors (14 low-grade and 3 high-grade appendiceal neoplasms, the remaining were not classified) and in one case the primary tumor was a mucinous adenocarcinoma of the ovary. The median PCI was 23.5 (min-max, 2-39). For 23 patients, complete cytoreduction was achieved (CC0/1) and these patients received mitomycin C-based HIPEC, while in 5 cases palliative procedures were performed and no HIPEC was given.

### Histopathology and Next-Generation DNA Sequencing

Fresh tumor samples were collected at the time of surgery from 22/25 cases. Samples were immediately snap frozen and stored at −80° C. until further processing. The tumor content was assessed in hematoxylin and eosin (H&E) stained frozen sections. Regardless of tumor content, available samples were homogenized and disrupted using TissueLyzer LT (Qiagen, Hilden, Germany). DNA was extracted using the AllPrep DNA/RNA/miRNA Universal Kit, automated with the use of QIAcube (Qiagen). For 10 PMP cases, no fresh tumor tissue was collected at CRS-HIPEC, (n=3) or no mutation was detected in fresh-frozen samples with no or very low tumor cell content (n=7). In these cases, DNA was additionally extracted from the formalin fixed, paraffin-embedded routine pathology samples of the peritoneal disease or the primary appendiceal tumor after micro dissection using the QIAcube and AllPrep DNA/RNA FFPE Kit. DNA purity was measured using NanoDrop (Thermo Fisher Scientific, Waltham, MA, USA); median absorbance ratio 260/280 was 1.83 (min-max 1.51-2.55), and concentrations were determined with the Qubit fluorometer (Thermo Fisher). Targeted next-generation sequencing (NGS) was performed with the Ion GeneStudio S5 system and the Oncomine Comprehensive Assay v3 (Thermo Fisher), covering single-nucleotide variants (SNVs) and indels from 161 unique genes. The median coverage of called variants was 4929, enabling detection of variants down to 1% allele frequency. Variants were called, annotated and filtered with Ion Reporter Software 5.10 (Thermo Fisher), and manually reassessed using Integrative Genomics Viewer. In DNA from the formalin-fixed samples, the presence of GNAS mutations was assessed using the ddPCR system from BioRad (BioRad®, Hercules, CA, USA). The ddPCR™ Mutation Assay (GNAS R201H (assayID: dHsaMDV2516796) and GNAS R201C (assayID: dHsaMDV2510562)) were used for detection of R201C and R201H mutations, respectively. Pre-mix preparation, droplet generation, and thermal cycling were performed according to the manufacturer's instructions. The fluorescence intensity in droplets was detected by a QX200 Droplet Reader (BioRad). For both assays, a “no template control” and a positive control were included for quality control. QuantaSoft version 1.7.4 analysis software and QX Manager Software (BioRad) was used for data acquisition and analysis. Only tests providing >13.000 droplets were considered valid.

### T-Cell Proliferation Assays

Peripheral blood mononuclear cells (PBMCs) from PMP patients and healthy donors were isolated and frozen and later thawed for testing as previously described [15]. PBMCs were stimulated once in vitro with 10 μM Gsα mutated peptides at 2×10⁶ cells/ml in X-Vivo 15 medium (Lonza, Basel, Switzerland), adding 20 U/ml IL-2 (Clinigen, Burton upon Trent UK) and 5 ng/ml IL-7 (Bio-Techne, Minneapolis, MN, USA) on day 3. The Gsα peptides (aa 186-215) contained point mutations R201H and R201C (ProImmune Ltd, Oxford, UK) and were of indicated lengths (30 amino acids). After 12-14 days, cells were tested in proliferation assays; pre-stimulated T cells were seeded at 5×10⁴ cells per well 96-well round-bottomed plates. The same number of irradiated (30 Gy), autologous PBMCs was added for use as antigen presenting cells (APCs) and Gsα mutated peptides as well as the wild-type peptide sequence were added at 10 μM. Staphylococcal superantigen SEC-3 (0.1 μM) was added as a positive control. Proliferation was measured at day 3 after labelling with 3.7×10⁴ Bq 3H-Thymidine (Montebello Diagnostics AS, Oslo, Norway) overnight before harvesting. All conditions were tested in triplicates. The Stimulatory Index (SI) was defined as proliferation with peptide divided by proliferation without peptide and standard deviations were calculated. SI≥2 was considered as a positive response.

CyTOF Analysis of Immune Cells from PMP Tissues

Fresh tumor tissue was disaggregated with Collagenase II (Sigma-Aldrich, St Louis, MI, USA) and DNase I (Sigma-Aldrich) after cutting tumor into small pieces. The single cell suspension was then washed and erythrocytes lysed by adding ACK Lysis Buffer (Thermo Fisher, Waltham, MA, USA), before a second wash. The single cells were frozen in fetal bovine serum (Gibco, Thermo Fisher Scientific, Waltham, MA USA) containing 12% DMSO (Sigma-Aldrich) and stored in liquid nitrogen. Briefly, single cells from biopsies were thawed, washed and resuspended in MaxPar cell staining buffer (Fluidigm, San Francisco, CA, USA), before staining with Cell-ID cisplatin (Fludigm) for 5 min, then washed and stained with extracellular antibodies for 30 min. The samples were then fixed with 1.6% paraformaldehyde and permeabilised in 99% methanol (Sigma-Aldrich). Samples were stored at −80° C. in methanol for up to 4 weeks. After removing methanol, all samples were incubated with iridium cell tracker (Fluidigm) for 20 min, washed and resuspended in water with 10% of calibration beads (Fluidigm). Samples were filtered immediately before sample acquisition on a CyTOF 2 (Fluidigm) instrument at the OUS Flow Cytometry core facility.

Analysis was performed using Cytobank Cellmass (cytobank.org). A panel of 35 extracellular markers was used. Typical gating strategy was applied as follows: 1. EQ-140 vs Ir-191 to exclude calibration beads. 2. Ir-191 vs Ir-193 to gate singlets. 3. Ir-191 vs event length to gate intact singlet. 4. Cisplatin vs CD45-89Y to gate live lymphocytes. All further analyses were carried out on this population. Cellular sub-populations were clustered and visualized using the viSNE (visual stochastic network embedding) analysis tool (based on the t-Distributed Stochastic Neighbor Embedding (t-SNE) clustering algorithm). The clustering was based on the expression of CD45RA, CD19, CD11b, CD4, CD20, CD21, IgD, CD14, CD8a, CD3, HLA-DR, CD56, CD16, CCR6, CD25, PD-L1, PD-L2, LAG-3, TIM-3, TIGIT, PD-1, CCR7, CD28, CTLA-4, ICOS, CXCR3, CXCR5, CXCR4, CD161, CD127, NKG2D, CD38, and CD33 and ran with the following parameters: and ran with the following parameters: 1000 iterations, 30 perplexity and 0.5 theta.

### Statistical Analysis

Paired, two-tailed t-tests were used to compare T cell proliferation against the wild-type Gsα peptide with each of the other conditions. All statistical analyses were performed using GraphPad Prism v.8 software (GraphPad Software, San Diego, CA, USA).

### Example 1—Tumor Mutation Analysis

Tumor samples from PMP patients were flash frozen as described above. Tumor content was analysed using H&E staining of frozen sections, and next generation sequencing was carried out on DNA extracted from on flash frozen tissue as described above. GNAS mutations were detected in samples from 22/25 patients (88%), while in three cases no mutation was found. The R201H and R201C mutations were detected in 16 and 6 cases, respectively (Table 2).

TABLE 2
Clinicopathological data, mutation status and proliferation score for PMP cases

| | | | | | | | Proliferation score | | |
|---|---|---|---|---|---|---|---|---|---|
| | | | | | | GNAS | according to peptide | | |
| Case# | Sex | Age | Primary tumor | PCI | HIPEC | mutation | Wild-type | R201C | R201H |
| 434 | M | 55 | Appendix | 32 | Yes | R201H | Not performed*** | | |
| 440 | F | 47 | Appendix | 3 | Yes | R201H | 1.5 | 4.4 | 5.7 |
| 445 | F | 70 | Appendix | 7 | Yes | R201C | 4.4 | 20.9 | 2.7 |
| 510 | F | 73 | Appendix | 27 | Yes | R201H | 2.9 | 7.4 | 18.8 |
| 519 | F | 60 | Appendix | 2 | Yes | R201C | 0.9 | 1.3 | 1.2 |
| 522 | M | 38 | Appendix | 11 | Yes | R201H | 1.0 | 0.9 | 1.6 |
| 523 | F | 61 | Appendix | 39 | No | NMD | 0.6 | 2.9 | 1.6 |
| 530 | F | 56 | Appendix | 38 | No | R201C | 9.4 | 16.8 | 16.1 |
| 534 | M | 48 | Appendix | 10 | Yes | R201H | 1.6 | 53.4 | 9.4 |
| 537 | F | 39 | Ovary | 29 | Yes | R201C | 24.6 | 58.8 | 46.6 |
| 543 | F | 72 | Appendix | 28 | Yes | R201H | 3.2 | 5.2 | 27.0 |
| 547 | M | 48 | Appendix | 39 | Yes | R201H | 1.1 | 1.1 | 0.6 |
| 549 | M | 32 | Appendix | 13 | Yes | R201H | 0.9 | 4.6 | 0.5 |
| 558 | F | 37 | Ovary | 6 | Yes | R201H | 1.4 | 1.0 | 0.2 |
| 559 | M | 54 | Appendix | 24 | Yes | NMD | 5.4 | 35.4 | 0.3 |
| 563 | F | 50 | Appendix | 23 | Yes | R201H | 6.2 | 15.3 | 10.4 |
| 570 | F | 70 | Appendix | 2 | Yes | R201H | 1.1 | 16.8 | 13.6 |
| 572 | M | 46 | Appendix | 30 | Yes | R201C | 2.4 | 10.2 | 28.3 |
| 575 | M | 72 | Appendix | 35 | No | R201C | 0.9 | 1.1 | 1.2 |
| 576 | F | 55 | Appendix | 19 | Yes | R201H | 1.0 | 4.7 | 3.5 |
| 581 | M | 39 | Appendix | 37 | Yes | R201H | 0.6 | 2.3 | 0.7 |
| 583 | F | 74 | Appendix | 21 | Yes | NMD | 0.8 | 1.1 | 1.1 |
| 585 | F | 53 | Appendix | 25 | Yes | R201H | 1.4 | 0.6 | 3.8 |
| 586 | F | 69 | Appendix | 32 | No | R201H | 1.1 | 1.0 | 2.2 |
| 588 | F | 59 | Appendix | 32 | No | R201H | 1.0 | 0.7 | 2.1 |

* No mutation detected, no tumor cells in sample
**A distinct variant detected, but number of reads below threshold
***Concordance between detected GNAS mutation and highest peptide response
NA; Not applicable.
PCI, peritoneal cancer index;
CC score, completeness of cytoreduction;
CRS-HIPEC, cytoreductive surgery and hyperthermic intraperitoneal chemotherapy

### Example 2—Peptide Immunogenicity

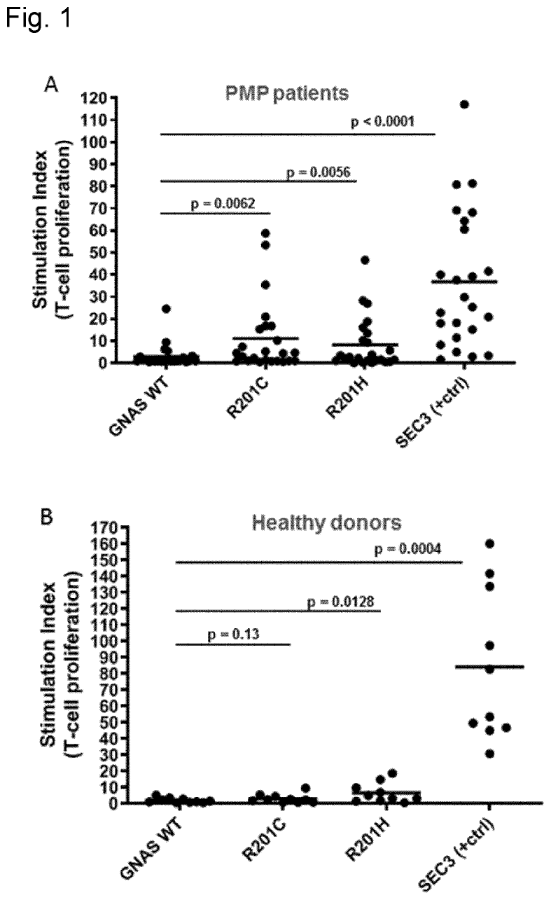

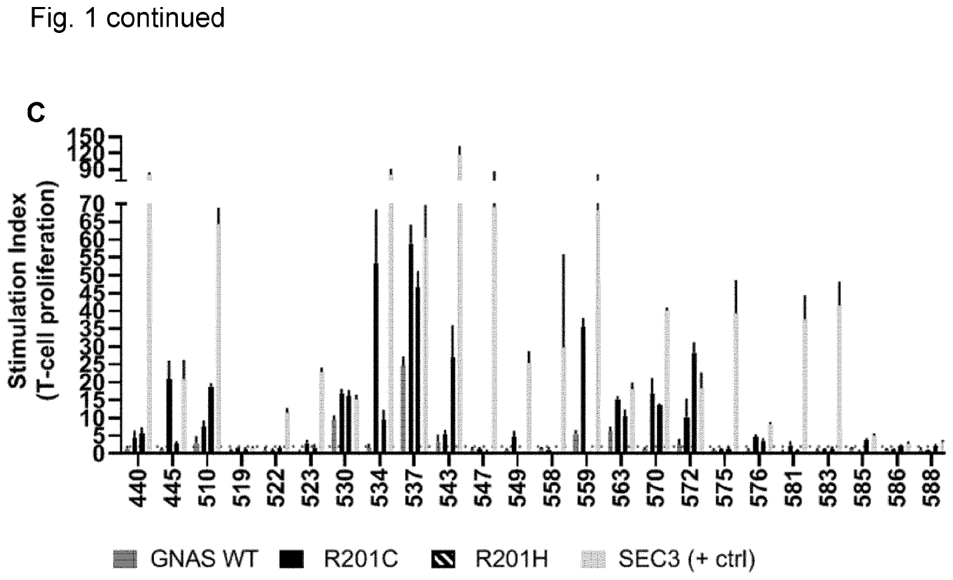

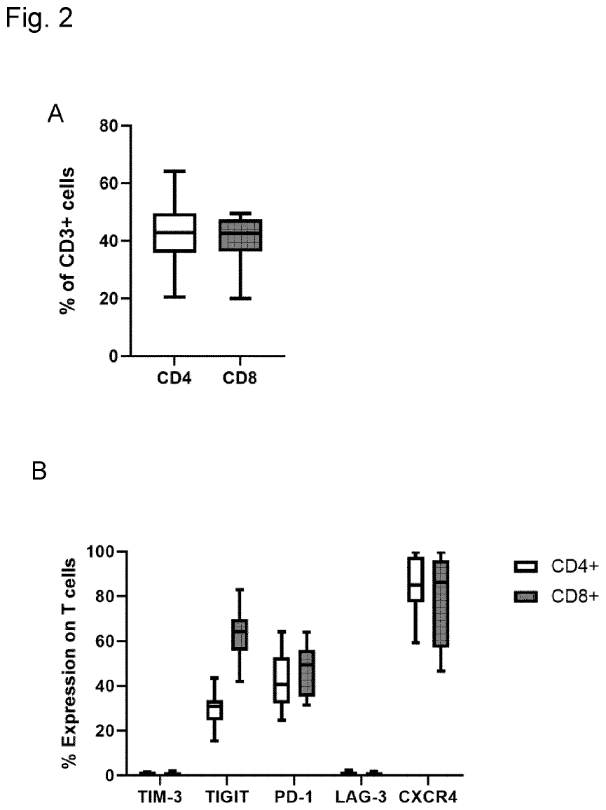

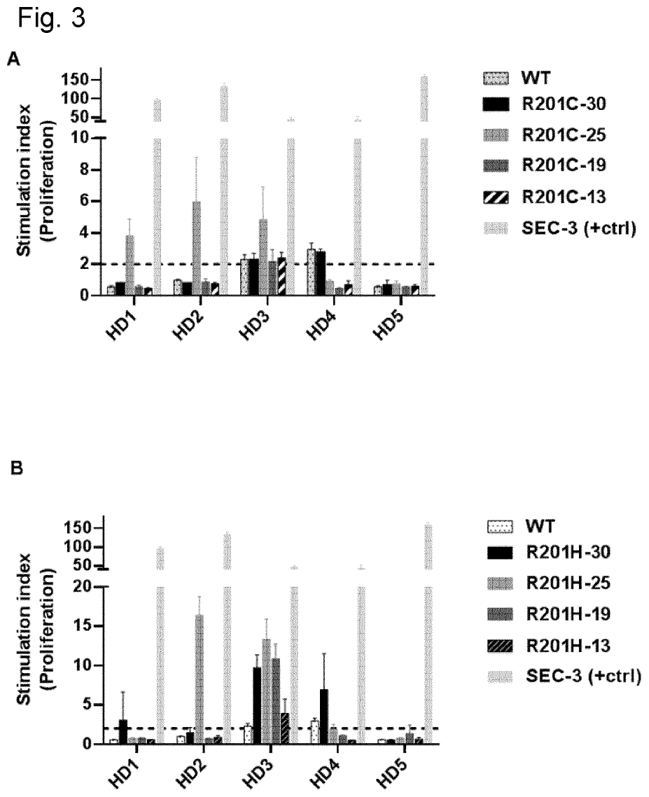

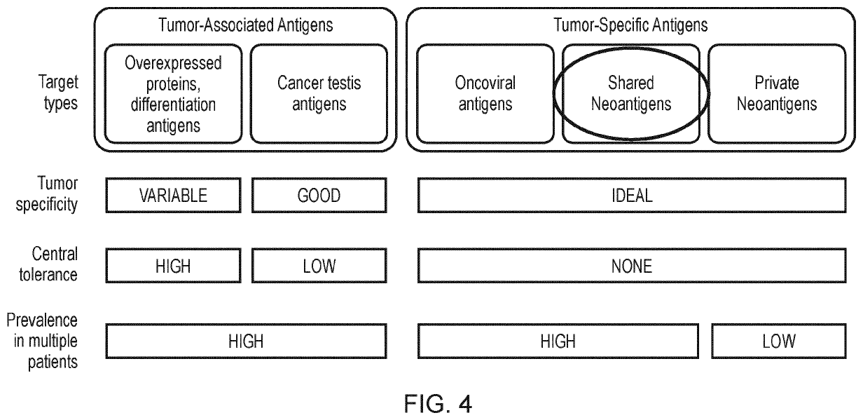

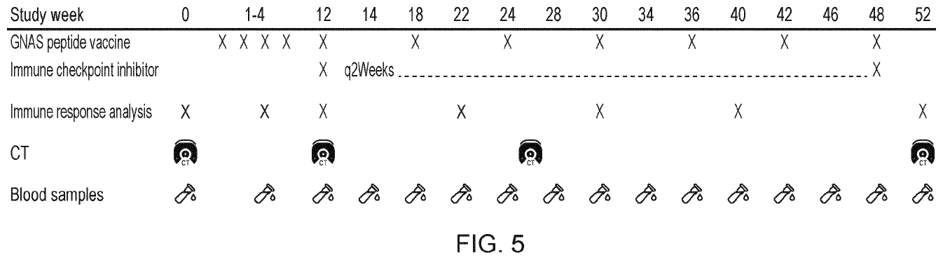

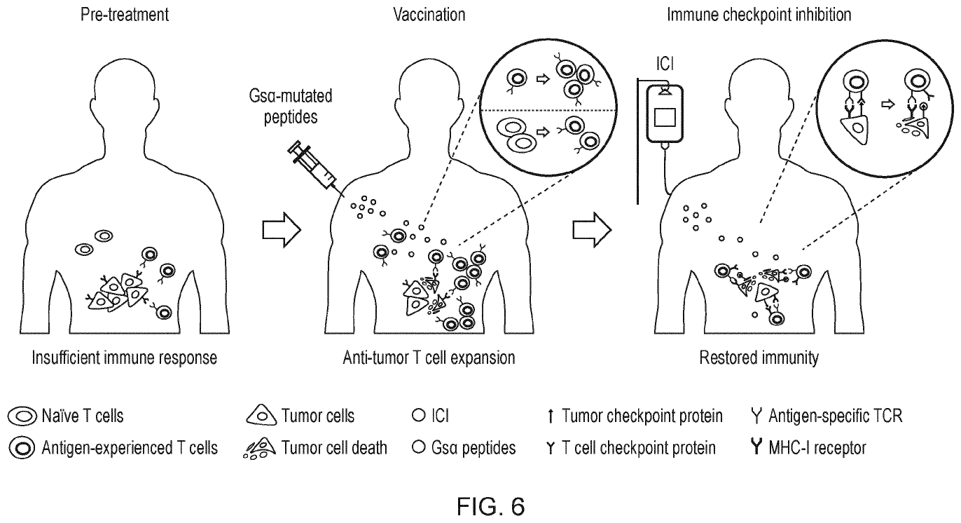

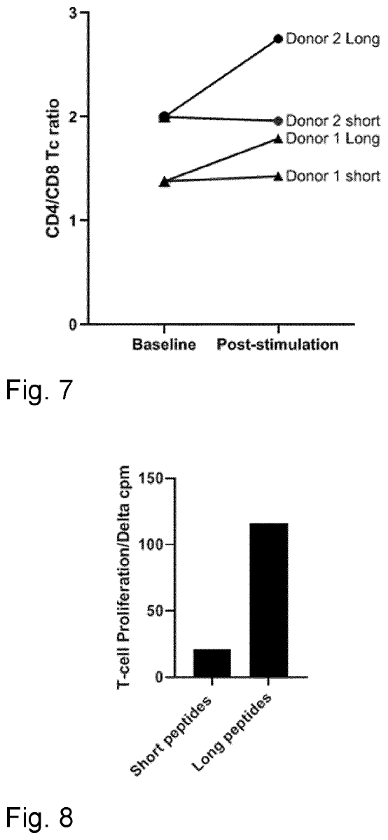

It was explored whether peptides with GNAS mutations are immunogenic. Immune responses against GNAS were tested in blood samples from 24 of the 25 patients and from 10 healthy donors. When stimulated with the mutated peptides, proliferative T-cell responses against one or both peptides were observed in 18 of 24 PMP cases (FIG. 1A, C and Table 2). A trend towards a preferential response against one of the peptides (proliferation value >20% larger than towards the other peptide) was noted in 15 of the 18 responding cases; 9 and 6 samples favoring the R201C and R201H peptides, respectively. The wild-type peptide also elicited responses in the PMP samples in 8 of the 24 samples analyzed, but in all cases, the response to one or both mutant peptides was stronger. Concordance between the mutation detected in the tumor samples and a preferential response towards the corresponding peptide was observed in 7 cases of the 15 cases where a preferential response was noted. For the healthy donors, responses were noted in 7 of the 10 analyzed samples (FIG. 1B). The responses in healthy donors were generally of lower magnitude than in PMP patients. As T cells from healthy donors are not thought to be primed against mutated GNAS in vivo the detection of GNAS-specific T-cell responses in these samples demonstrates the strong immunogenicity of these peptides, although this was not significant compared to wt GNAS.

The response against mutated GNAS peptides in healthy donors was lower than in PMP patients. Nevertheless, the induction of a T cell response even in healthy donors demonstrates that these peptides are very immunogenic. In patient #445, the R201C mutation was detected and, interestingly, in this case the response to the corresponding peptide was dramatically more pronounced than to the other peptide, also suggesting a specific pre-existing immune response.

The findings are extremely interesting, since they offer a completely new view of the immunology and pathogenesis of PMP. Since these patients have all developed clinical PMP, the immune response must by definition have been inhibited as part of the pathogenic process. This again suggests that the immune activity could contribute to long-term disease control.

### Example 3—Tumour Infiltrates in PMP

Very little is known about immune cell infiltration and the tumor microenvironment (TME) in PMP. In tumor biopsies from patients with PMP and non-PMP peritoneal metastases immune profiling by mass cytometry (CyTOF) was performed to investigate whether the tumor tissue contains immune cells that could be reactivated by immunotherapy (FIG. 2). Successful preparation of single cell suspensions from PMP samples with subsequent staining and analysis by mass cytometry (CyTOF) was achieved in 18/25 cases. The results clearly showed that most PMP patients had CD3+ T cell infiltration. The CD4:CD8 T cell ratio was variable and in some cases did not make up 100% of the CD3+ population. Some of these CD3+ cells could be CD8-CD4-NKT cells or double negative T cells which have also been described to be immune suppressive.

A large part of the infiltrating T cells seemed, however, to be antigen-experienced as they expressed varying levels of immune checkpoint inhibitor molecules. The most predominantly expressed checkpoints were PD-1 and TIGIT, whereas TIM-3 and LAG-3 were seen at very low levels in a few patients. PD-1 levels were similarly expressed by CD4+ and CD8+ T cells with an average expression of 40-50%. In contrast, TIGIT was predominantly expressed by CD8+ T cells (average of 60%), whereas the average expression on CD4+ T cells was around 35%, indicating that these T cells have seen their cognate antigen in vivo [3]. Some T cell populations expressed both TIGIT and PD-1 checkpoint molecules. Additionally, the T cells expressed high levels of the chemokine receptor CXCR4, which is linked to T cell receptor (TCR) signaling and T-cell exhaustion [4] and may contribute to T cell homing to tumor. CXCR4 expression has been implicated in metastasis of several cancers, including in colorectal cancer [23], but is also important in lymphocyte trafficking. Its ligand-stromal cell-derived factor-1 (SDF1, CXCL12) is produced in the tumor microenvironment [24]. This indicates that it is possible to mount an anti-tumor immune response, and that these T cells can survive in the TME in PMP patients, two central properties required for a vaccine to work.

Our results show that infiltrating lymphocyte populations can be found even in mucinous tumors such as PMP. PMP therefore seems to be immunogenic which is in line with the peripheral immune responses we see in patient blood samples against mutated GNAS. Analysis was focused on T cell subsets, since T cells can recognize specific neo-antigens and would be the target cells for a peptide vaccine. Mass cytometry analysis showed that both CD4+ and CD8+ T cells infiltrated the PMP biopsies, and both T cell populations expressed PD-1 and TIGIT checkpoint receptors suggesting that these T cells are antigen experienced and likely tumor reactive. The detection of strong T cell responses against mutated Gsα peptides (R201C or R201H) in the circulation of the majority of patients screened indicates that such antigen priming has indeed taken place in patients. On the one hand, PD-1 receptor engagement has an inhibitory effect on T cell effector functions and high levels of PD-1 have been associated with T cell exhaustion and a dysfunctional phenotype. The inhibition can be caused by tumor intrinsic mechanisms or by cells or factors in the tumor microenvironment such as macrophages.

Therapeutic vaccination with GNAS peptides will lead to enhanced anti-tumour responses, killing of tumour cells and spreading of the immune response to other tumour antigens. A broad immune response after vaccination has been shown to correlate with clinical efficacy of vaccination [5]. Numerous previous cancer vaccines were based on short peptides without optimized adjuvants and were tested in patients with very advanced disease, yielding disappointing clinical results. The situation is different for PMP patients, where the advantage is that we can vaccinate after tumour debulking or elimination by surgery, when relapses generally take time, but remain incurable. This has been shown to be an ideal situation for therapeutic cancer vaccination [6].

Notably, the inventors have identified that this tumour antigen, mutated GNAS, is an ideal antigen to target with therapeutic vaccination (FIG. 4): as it has high tumour specificity as it is mutated only in cancer cells, there is no immune tolerance against it as is the case with self-antigens expressed elsewhere, but overexpressed in tumour, and because it is a frequent mutation in this patient group it is does not have to be individually adapted for each patient as is the case for private neoantigens. GNAS is also considered to be a driver oncogene and its expression is therefore not likely to be easily lost by the tumor cells.

### Example 4—Combination with Checkpoint Inhibitors

More recently developed immune checkpoint inhibitors, especially PD-1/PD-L1 antagonists, offer key opportunities for maximizing the efficacy of therapeutic cancer vaccines. Checkpoint inhibitors (CPI) depend on existing spontaneous immune responses in the patient for effect and therefore do not work in patients lacking those. Multiple preclinical studies have shown synergy between therapeutic vaccination and CPIs, and several clinical studies are now evaluating this.

Indeed, a recent study reported circulating CD8+ T-cells double positive for PD-1 and TIGIT to be an early marker of therapeutic response to anti-PD-1 therapy in melanoma and Merkel cell carcinoma [25]. From the perspective of currently known predictors of response to immune checkpoint inhibitors, PMP is not an ideal candidate for CPI monotreatment. In a total of 183 investigated in 6 individual studies, only one case was identified as microsatellite instability-high (MSI) [16-21]. However, the results from CyTOF analyses of tumor infiltrating immune cells show that in a majority of PMP patients, T-cells express immune checkpoint molecules such as PD-1, TIGIT, and TIM-3. This indicates that the T-cells have already been activated in an antigen-specific way. To enhance this pre-existing intra-tumoral immune response by combination with well-established anti-PD-1/PD-L1 therapy or more novel checkpoint inhibitors can provide clinical benefit, offering a curative treatment to PMP patients and for patients suffering from other GNAS-mutated cancers.

Previously activated T cells that become exhausted and express immune checkpoint molecules can be detected in biopsies; hence they survive in the TME, but are likely inhibited. This inhibition can be directly by tumor intrinsic mechanisms or by cells or factors in the TME such as macrophages which were found to be abundant in the majority of the PMP patients. This clearly provides a rationale for combining vaccination with immune checkpoint inhibitor treatment. It is likely that some T cell populations express multiple checkpoint molecules. This suggests that PMP is immunogenic and can activate T cells that become exhausted and inhibited, but our results show that they survive in the TME. This also provides a rationale for combining vaccination with immune checkpoint inhibitor treatment, in particular PD-1 or PD-1 inhibition.

We will carry out a signal-finding, prospective, open label phase I trial, (one fixed dose; 3+3 design) at Oslo University Hospital as follows:

Study participants: 10 Main inclusion criteria: Adult (age 18 years or older) patients with recurrent or non-resectable GNAS mutated

PMP Study hypothesis: The study hypothesis can be seen represented in FIG. 6. In particular, patients with GNAS-mutated PMP has a pre-existing, attenuated immune response directed against mutated GNAS protein. Vaccination with mutated GNAS peptides will be well tolerated alone and can be combined with immune checkpoint inhibition with acceptable toxicity. The treatment will cause reactivation of the immune response and immune checkpoint inhibition will restore measurable anticancer immunity in patients.

### Study Interventions: (See FIG. 5)

1. Mutated GNAS peptide vaccine+adjuvant (GM-CSF) will be administered on days 1, 8, 15 and 22. Five subsequent boost vaccinations with will be administered at 6 week intervals (on weeks 12, 18, 24, 30, 42, and 48).
2. Immune checkpoint inhibitor will be administered at the recommended dosing schedule, for instance every 2 weeks for a total treatment period of one year.

### Primary Outcome Measures:

1. Number of study participants experiencing study-related drug toxicities; number of participants experiencing study drug-related adverse events Grade 3 or higher as defined by CTCAE v5.0.
2. Immune response against vaccine, measured as increase in interferon-gamma (IFN-γ) production or proliferation of vaccine-specific T-cells at 8, 12, 22, 30, 40, and 52 weeks compared to pre-vaccination baseline. IFN-γ production will be measured by ELISPOT and T-cell proliferation by 3H-thymidine incorporation. The T-cells will be pre-stimulated one round in vitro with mutant GNAS peptides in order to increase the frequency of GNAS-specific T cells prior to testing, as the level of circulating vaccine-specific T cells may be below detection limit in direct testing of blood samples.

### Secondary Outcome Measures:

Number of months from date of first treatment until disease progression (assessed by abdominopelvic CT scans using modified RECIST 2.0 and/or blood levels of carcinoembryonic antigen (CEA) and/or detection of GNAS mutation by ctDNA analysis) to determine progression-free survival (PFS).

### Example 5

### Method:

### Flow Cytometry

Peripheral blood mononuclear cells (PBMCs) from healthy donors were isolated and frozen and later thawed for testing as previously described [15]. PBMCs were stimulated once in vitro with peptide mixtures containing 10 μM of each of the Gsα mutated peptides at 2×10⁶ cells/ml in CellGro Dc medium (CellGenix GmbH, Freiburg, Germany), adding 20 U/ml IL-2 (Clinigen, Burton upon Trent UK) and 5 ng/ml IL-7 (Bio-Techne, Minneapolis, MN, USA) on day 3. The Gsα peptides contained point mutations R201H and R201C and were 30 amino acids long (SEQ ID NOs: 1 and 2, referred to as the “long peptides”; aa 186-216 of GNAS) or were 13, 14 or 12 amino acids long (SEQ ID NOs: 12-15, respectively, referred to herein as the “short peptides”), as shown in Table 3. The short peptides (SEQ ID NOs: 13-15) are peptides 3, 8 and 11, respectively, of CN 111072763. All peptides were produced by ProImmune Ltd, Oxford, UK.

TABLE 3

| | | | SEQ |
|---|---|---|---|
| Peptide | Peptide | | ID |
| mix | name | Sequence | NO |
| Short | Pep3 | DQDLLRCCVLTSG | 13 |
| peptides | Pep8 | QDLLRCHVLTSGIF | 14 |
| | Pep11 | LLRCHVLTSGIF | 15 |
| Long | GNAS | KQADYVPSDQDLLRCHVLTSGIFETKFQVD | 2 |
| peptides | R201H | | |
| | GNAS | KQADYVPSDQDLLRCCVLTSGIFETKFQVD | 1 |
| | R201C | | |

After 12-14 days, cells were stained with antibodies for flow cytometry analysis of CD4/CD8 ratios. New baseline (non-stimulated PBMC) samples were thawed, and PBMCs and pre-stimulated T cells were stained from each donor for comparison.

T cells were washed in flow cytometry staining buffer; phosphate buffered saline (PBS) solution supplemented with 2% fetal bovine serum (FBS)(Thermo Fisher Scientific, Waltham, MA, USA), then stained with anti-CD3 (OKT3), anti-CD4 (RPA-T4), and anti-CD8 (RPA-T8), all from BD Biosciences, San Jose, CA, USA. Cells were then washed in staining buffer prior to direct acquisition on a FACS Canto II (BD Biosciences). Data were analyzed using FlowJo software (Treestar Inc., Ashland, USA).

### T-Cell Proliferation Assays

Peripheral blood mononuclear cells (PBMCs) from healthy donors were isolated and frozen and later thawed for testing as previously described [15]. PBMCs were stimulated once in vitro with peptide mixtures containing 10 μM of each of the Gsα mutated peptides at 2×10⁶ cells/ml in CellGro Dc medium (CellGenix GmbH, Freiburg, Germany), adding 20 U/ml IL-2 (Clinigen, Burton upon Trent UK) and 5 ng/ml IL-7 (Bio-Techne, Minneapolis, MN, USA) on day 3. The peptide mixture and Gsα peptides were the same as used for the flow cytometry (see Table 3 above). All peptides were produced by ProImmune Ltd, Oxford, UK.

After 12-14 days, cells were tested in proliferation assays: pre-stimulated T cells were seeded at 5×10⁴ cells per well 96-well round-bottomed plates. The same number of irradiated (30 Gy), autologous PBMCs was added for use as antigen presenting cells (APCs) and Gsα mutated peptides were added at 10 μM. Proliferation was measured at day 3 after labelling with 3.7×10⁴ Bq 3H-Thymidine (Montebello Diagnostics AS, Oslo, Norway) overnight before harvesting. All conditions were tested in triplicates. The proliferation was read as count per minute (cpm) of the radioactivity and the difference (delta value) between the background and the T cells stimulated with the short peptides or the long peptides was calculated.

### Results:

It is known in the field that CD4 T helper cells are required to promote the activity of cytotoxic CD8 T lymphocytes (CTL, “killer cells”). CD8 T cells, or CTLs, recognise their cognate peptide on HLA class I molecules, generally in peptides that are 8-14 amino acids long [26]. CD4 T cells recognise peptides presented on HLA class II molecules, and the epitopes recognised generally have a length of 12-19 amino acids [27]. An effective vaccine should induce CD4 T cells, and the present inventors have appreciated that with longer peptides one is sure that HLA class II from a broad population can bind peptides and present these to CD4 T cells which are required to induce a full immune activation, including a helper T cell function for CD8 T cells. In the present Example, non-HLA-typed healthy donors were used and the results show that stimulating PBMCs from these donors with long peptides (30mers) generate more CD4 T cells than when shorter peptides (12-14mers) are used, thereby increasing the CD4/CD8 ratio (FIG. 7). The present Example also shows that the long peptides are more immunogenic than the short peptides (FIG. 8).

Recently, the important roles of CD4 T cells have been increasingly recognised in the cancer therapy field and these are considered crucial for a successful anti-tumour response [28-31]. Previous clinical cancer vaccine trials using long peptides have always seen a correlation between the proliferation and activation of CD4 T cells and patient survival [32-37]. Therefore, the identification of novel tumour-specific peptides that can induce more CD4 T cell activation expands the number of attractive targets for immunotherapy of cancer. The present Example shows that the mutated GNAS peptides of the present invention preferentially induce CD4 T cells that proliferate, such that these peptides are expected to have an advantage in the treatment of cancer.

### REFERENCES

1) Pengelly, R. J. et al. The Journal of molecular diagnostics: JMD 20, 635-642, doi:10.1016/j.jmoldx.2018.05.002 (2018).
2) Bailey, M. H. et al. Cell 174, 1034-1035, doi:10.1016/j.cell.2018.07.034 (2018).
3) Chauvin, J. M. et al. The Journal of clinical investigation 125, 2046-2058, doi:10.1172/jci80445 (2015).
4) Ramonell, K. M. et al. PloS one 12, e0188882, doi:10.1371/journal.pone.0188882 (2017).
5) Inderberg-Suso, E. M., Trachsel, S., Lislerud, K., Rasmussen, A. M. & Gaudernack, G. Oncoimmunology 1, 670-686, doi:10.4161/onci.20426 (2012).
6) van Willigen, W. W. et al. Frontiers in immunology 9, 2265, doi:10.3389/fimmu.2018.02265 (2018).
7) Hollingsworth, R. E. & Jansen, K. NPJ vaccines 4, 7, doi:10.1038/s41541-019-0103-y (2019).
8) Morgan, K. K. Cancer Today 01 (2019). (https://www.cancertodaymag.org).
9) Miller, K. L. Orphanet journal of rare diseases 12, 114, doi:10.1186/s13023-017-0665-6 (2017).
10) Innamorati G, Wilkie™, Kantheti H S, et al. BMC Cancer. 18(1):293 (2018).
11) O'Hayre M, Vazquez-Prado J, Kufareva I, et al. Nat Rev Cancer. 13(6):412-424.
(2013)
12) Pietrantonio F, Perrone F, Mennitto A, et al. Toward the molecular dissection of peritoneal pseudomyxoma. Ann Oncol. 2016; 27(11):2097-103.
13) Pietrantonio F, Berenato R, Maggi C, et al. GNAS mutations as prognostic biomarker in patients with relapsed peritoneal pseudomyxoma receiving metronomic capecitabine and bevacizumab: a clinical and translational study. J Transl Med. 2016; 14(1):125.
14) Sugarbaker, P. H. (2009). “Epithelial appendiceal neoplasms.” Cancer J 15(3): 225-235
15) Inderberg-Suso, E. M., S. Trachsel, K. Lislerud, A. M. Rasmussen and G. Gaudernack (2012). “Widespread CD4+ T-cell reactivity to novel hTERT epitopes following vaccination of cancer patients with a single hTERT peptide GV1001.” Oncoimmunology 1(5): 670-686.
16) Kabbani, W., P. S. Houlihan, R. Luthra, S. R. Hamilton and A. Rashid (2002). “Mucinous and nonmucinous appendiceal adenocarcinomas: different clinicopathological features but similar genetic alterations.” Mod Pathol 15(6): 599-605.
17) Zauber, P., E. Berman, S. Marotta, M. Sabbath-Solitare and T. Bishop (2011). “Ki-ras gene mutations are invariably present in low-grade mucinous tumors of the vermiform appendix.” Scand J Gastroenterol 46(7-8): 869-874.
18) Nummela, P., L. Saarinen, A. Thiel, P. Jarvinen, R. Lehtonen, A. Lepisto, H. Jarvinen, L. A. Aaltonen, S. Hautaniemi and A. Ristimaki (2015). “Genomic profile of pseudomyxoma peritonei analyzed using next-generation sequencing and immunohistochemistry.” Int J Cancer 136(5): E282-289.
19) Borazanci, E., S. Z. Millis, J. Kimbrough, N. Doll, D. Von Hoff and R. K. Ramanathan (2017). “Potential actionable targets in appendiceal cancer detected by immunohistochemistry, fluorescent in situ hybridization, and mutational analysis.” J Gastrointest Oncol 8(1): 164-172.
20) Gleeson, E. M., R. Feldman, B. L. Mapow, L. T. Mackovick, K. M. Ward, W. F. Morano, R. R. Rubin and W. B. Bowne (2018). “Appendix-derived Pseudomyxoma Peritonei (PMP): Molecular Profiling Toward Treatment of a Rare Malignancy.” Am J Clin Oncol 41(8): 777-783.
21)Tokunaga, R., J. Xiu, C. Johnston, R. M. Goldberg, P. A. Philip, A. Seeber, M. Naseem, J. H. Lo, H. Arai, F. Battaglin, A. Puccini, M. D. Berger, S. Soni, W. Zhang, J. J. Hwang, A. F. Shields, J. L. Marshall, H. Baba, W. M. Korn and H. J. Lenz (2019). “Molecular Profiling of Appendiceal Adenocarcinoma and Comparison with Right-sided and Left-sided Colorectal Cancer.” Clin Cancer Res 25(10): 3096-3103.
22) Bastepe M. Relative functions of Gas and its extra-large variant XLas in the endocrine system. Horm Metab Res. 2012 September; 44(10):732-40. doi: 10.1055/s-0032-1316331. PMID: 22730013.
23) Stanisavljevic, L., J. Assmus, K. E. Storli, S. M. Leh, O. Dahl and M. P. Myklebust (2016). “CXCR4, CXCL12 and the relative CXCL12-CXCR4 expression as prognostic factors in colon cancer.” Tumour Biol 37(6): 7441-7452.
24) Smit, M. J., G. Schlecht-Louf, M. Neves, J, v. d. Bor, P. Penela, M. Siderius, F. Bachelerie and F. MayorJr. (2021). “The CXCL12/CXCR4/ACKR3 Axis in the Tumor Microenvironment: Signaling, Crosstalk, and Therapeutic Targeting.” Annual Review of Pharmacology and Toxicology 61(1): 541-563.
25) Simon, S., V. Voillet, V. Vignard, Z. Wu, C. Dabrowski, N. Jouand, T. Beauvais, A. Khammari, C. Braudeau, R. Josien, O. Adotevi, C. Laheurte, F. Aubin, C. Nardin, S. Rulli, R. Gottardo, N. Ramchurren, M. Cheever, S. P. Fling, C. D. Church, P. Nghiem, B. Dreno, S. R. Riddell and N. Labarriere (2020). “PD-1 and TIGIT coexpression identifies a circulating CD8 T cell subset predictive of response to anti-PD-1 therapy.” J Immunother Cancer 8(2).
26) Wooldridge L. Individual MHCI-Restricted T-Cell Receptors are Characterized by a Unique Peptide Recognition Signature. Frontiers in Immunology [Internet]. 2013 [cited 2022 May 6]; 4. Available from: https://www.frontiersin.org/article/10.3389/fimmu.2013.00199.
27) Holland C J, Cole D K, Godkin A. Re-Directing CD4(+) T Cell Responses with the Flanking Residues of MHC Class II-Bound Peptides: The Core is Not Enough. Front Immunol. 2013; 4:172.
28) Borst J, Ahrends T, Babala N, Melief C J M, Kastenmuller W. CD4(+) T cell help in cancer immunology and immunotherapy. Nat Rev Immunol. 2018 October; 18(10):635-47.
29) Tay R E, Richardson E K, Toh H C. Revisiting the role of CD4+ T cells in cancer immunotherapy-new insights into old paradigms. Cancer Gene Therapy. 2020 May 27; 1-13.
30) Cachot A, Bilous M, Liu Y C, Li X, Saillard M, Cenerenti M, et al. Tumor-specific cytolytic CD4 T cells mediate immunity against human cancer. Sci Adv. 2021 February; 7(9):eabe3348.
31) Poncette L, Bluhm J, Blankenstein T. The role of CD4 T cells in rejection of solid tumors. Curr Opin Immunol. 2022 February; 74:18-24.
32) Brunsvig P F, Aamdal S, Gjertsen M K, Kvalheim G, Markowski-Grimsrud C J, Sve I, et al. Telomerase peptide vaccination: a phase I/II study in patients with non-small cell lung cancer. Cancer immunology, immunotherapy: CII. 2006 December; 55(12):1553-64.
33) Bernhardt S L, Gjertsen M K, Trachsel S, Moller M, Eriksen J A, Meo M, et al. Telomerase peptide vaccination of patients with non-resectable pancreatic cancer: A dose escalating phase I/II study. Br J Cancer. 2006 Dec. 4; 95(11):1474-82.
34) Brunsvig P F, Kyte J A, Kersten C, Sundstrom S, Moller M, Nyakas M, et al. Telomerase peptide vaccination in NSCLC: a phase II trial in stage Ill patients vaccinated after chemoradiotherapy and an 8-year update on a phase I/II trial. Clin Cancer Res. 2011 Nov. 1; 17(21):6847-57.
35) Kyte J A, Gaudernack G, Dueland S, Trachsel S, Julsrud L, Aamdal S. Telomerase peptide vaccination combined with temozolomide: a clinical trial in stage I V melanoma patients. Clin Cancer Res. 2011 Jul. 1; 17(13):4568-80.
36) Lilleby W, Gaudernack G, Brunsvig P F, Vlatkovic L, Schulz M, Mills K, et al. Phase/IIIa clinical trial of a novel hTERT peptide vaccine in men with metastatic hormone-naive prostate cancer. Cancer Immunol Immunother. 2017 July; 66(7):891-901.
37) Brunsvig P F, Guren T K, Nyakas M, Steinfeldt-Reisse C H, Rasch W, Kyte J A, et al. Long-Term Outcomes of a Phase I Study With UV1, a Second Generation Telomerase Based Vaccine, in Patients With Advanced Non-Small Cell Lung Cancer. Frontiers in Immunology [Internet]. 2020 November; 11. Available from: https://doi.org/10.3389%2Ffimmu.2020.572172

Sequences

| SEQ ID NO: | Peptide name | Peptide sequence |
|---|---|---|
| 1 | GNAS R201C Full | kqadyvpsdqdllrccvltsgifetkfqvd |
| 7 | GNAS R201C-25 | dyvpsdqdllrccvltsgifetkfq |
| 5 | GNAS R201C-19 | psdqdllrccvltsgifet |
| 3 | GNAS R201C-13 | qdllrccvltsgi |
| 2 | GNAS R201H Full | kqadyvpsdqdllrchvltsgifetkfqvd |
| 8 | GNAS R201H-25 | dyvpsdqdllrchvltsgifetkfq |
| 6 | GNAS R201H-19 | psdqdllrchvltsgifet |
| 4 | GNAS R201H-13 | qdllrchvltsgi |
| 9 | GNAS WT | kqadyvpsdqdllrcrvltsgifetkfqvd |

SEQ ID NO: 10-Wild type GNAS amino acid sequence
MGCLGNSKTE DQRNEEKAQR EANKKIEKQL QKDKQVYRAT HRLLLLGAGE
SGKSTIVKQM RILHVNGFNG EGGEEDPQAA RSNSDGEKAT KVQDIKNNLK
EAIETIVAAM SNLVPPVELA NPENQFRVDY ILSVMNVPDF DFPPEFYEHA
KALWEDEGVR ACYERSNEYQ LIDCAQYFLD KIDVIKQADY VPSDQDLLRC
RVLTSGIFET KFQVDKVNFH MFDVGGQRDE RRKWIQCFND VTAIIFVVAS
SSYNMVIRED NQTNRLQEAL NLFKSIWNNR WLRTISVILF LNKQDLLAEK
VLAGKSKIED YFPEFARYTT PEDATPEPGE DPRVTRAKYF IRDEFLRIST
ASGDHRHYCY PHFTCAVDTE NIRRVFNDCR DIIQRMHLRQ YELL
SEQ ID NO: 11-Mutated R201C GNAS amino acid sequence found in patients
MGCLGNSKTE DQRNEEKAQR EANKKIEKQL QKDKQVYRAT HRLLLLGAGE
SGKSTIVKQM RILHVNGFNG EGGEEDPQAA RSNSDGEKAT KVQDIKNNLK
EAIETIVAAM SNLVPPVELA NPENQFRVDY ILSVMNVPDF DFPPEFYEHA
KALWEDEGVR ACYERSNEYQ LIDCAQYFLD KIDVIKQADY VPSDQDLLRC
CVLTSGIFET KFQVDKVNFH MFDVGGQRDE RRKWIQCFND VTAIIFVVAS
SSYNMVIRED NQTNRLQEAL NLFKSIWNNR WLRTISVILF LNKQDLLAEK
VLAGKSKIED YFPEFARYTT PEDATPEPGE DPRVTRAKYF IRDEFLRIST
ASGDGRHYCY PHFTCAVDTE NIRRVFNDCR DIIQRMHLRQ YELL
SEQ ID NO: 12-Mutated R201H GNAS amino acid sequence found in patients
MGCLGNSKTE DQRNEEKAQR EANKKIEKQL QKDKQVYRAT HRLLLLGAGE
SGKSTIVKQM RILHVNGFNG EGGEEDPQAA RSNSDGEKAT KVQDIKNNLK
EAIETIVAAM SNLVPPVELA NPENQFRVDY ILSVMNVPDF DFPPEFYEHA
KALWEDEGVR ACYERSNEYQ LIDCAQYFLD KIDVIKQADY VPSDQDLLRC
HVLTSGIFET KFQVDKVNFH MFDVGGQRDE RRKWIQCFND VTAIIFVVAS
SSYNMVIRED NQTNRLQEAL NLFKSIWNNR WLRTISVILF LNKQDLLAEK
VLAGKSKIED YFPEFARYTT PEDATPEPGE DPRVTRAKYF IRDEFLRIST
ASGDGRHYCY PHFTCAVDTE NIRRVENDCR DIIQRMHLRQ YELL

1-24. (canceled)

25. A peptide mixture suitable for eliciting an immune response, comprising a first peptide and a second peptide,
wherein the first peptide comprises a first sequence of at least 8 amino acids, wherein said first sequence has at least 75% sequence identity to a first region of 8 amino acids of SEQ ID NO: 11 wherein said first region includes position 201 of SEQ ID NO: 11 such that the first sequence includes the amino acid in position 201 of SEQ ID NO: 11, and
wherein the second peptide comprises a second sequence of at least 8 amino acids, wherein said second sequence has at least 75% sequence identity to a second region of 8 amino acids of SEQ ID NO: 12, and wherein said second region includes position 201 of SEQ ID NO: 12 such that the second sequence includes the amino acid in position 201 of SEQ ID NO: 12.

26. A peptide mixture according to claim 25, wherein each of the first and second peptides consists of no more than 100 amino acids.

27. A peptide mixture according to claim 25, wherein the amino acid in position 201 of SEQ ID NO: 11 in the first sequence is flanked on each side by at least two amino acids, and the amino acid in position 201 of SEQ ID NO: 12 in the second sequence is flanked on each side by at least two amino acids.

28. A peptide mixture according to claim 25, wherein the sequence of at least 8 amino acids in the first peptide has 100% sequence identity to the first region of 8 amino acids including position 201 of SEQ ID NO: 11 and the sequence of at least 8 amino acids in the second peptide has 100% sequence identity to the second region of 8 amino acids including position 201 of SEQ ID NO: 12.

29. A peptide mixture according to claim 25, wherein the first peptide comprises or consists of a sequence having at least 75% sequence identity to a sequence selected from SEQ ID NOs: 1, 3, 5 and 7, and the second peptide comprises or consists of a sequence having at least 75% sequence identity to a sequence selected from SEQ ID NOs: 2, 4, 6 and 8.

30. A peptide suitable for eliciting an immune response, selected from:
peptides that consist of a sequence having at least 75% sequence identity to SEQ ID NO: 1 or 2,
peptides that consist of a sequence having at least 85% sequence identity to SEQ ID NO: 7 or 8,
peptides that consist of a sequence consisting of 19 amino acids and having at least 95% sequence identity to SEQ ID NO: 5 or 6, and
peptides that consist of a sequence consisting of 13 amino acids and having at least 90% sequence identity to SEQ ID NO: 3 or SEQ ID NO: 4.

31. A peptide according to claim 30, wherein the peptide is selected from:
peptides that consist of a sequence having at least 80%, at least 85%, at least 90%, at least 95%, at least 97%, at least 98%, at least 99%, or 100% sequence identity to SEQ ID NO: 1 or 2,
peptides that consist of a sequence having at least 97%, at least 98%, at least 99% or 100% sequence identity to SEQ ID NO: 7 or 8,
peptides that consist of a sequence consisting of 19 amino acids and having at least 97%, at least 98%, at least 99% or 100% sequence identity to SEQ ID NO: 5 or 6, and
peptides that consist of a sequence consisting of 13 amino acids and having at least 95%, at least 97%, at least 98%, at least 99% or 100% sequence identity to SEQ ID NO: 3 or SEQ ID NO: 4.

32. A peptide according to claim 30, wherein the peptide consists of the amino acid sequence of SEQ ID NO: 3 or 4.

33. A T-cell mixture comprising T-cells specific for each of the peptides in one of the GC peptide mixtures according to claim 25, when presented on an MHC molecule

34. A T-cell preparation comprising a T-cell specific for a peptide according to claim 30, when presented on an MHC molecule.

35. A nucleic acid molecule encoding a peptide according to claim 30.

36. At least one nucleic acid molecule, wherein the nucleic acid molecule or molecules individually or collectively comprise nucleotide sequences encoding at least two of the peptides defined in claim 25.

37. A vector comprising a nucleic acid molecule according to claim 35.

38. A vector comprising at least one nucleic acid molecule according to claim 36.

39. A host cell comprising the vector of claim 37, optionally wherein said host cell is E. coli.

40. A host cell comprising the vector of claim 38, optionally wherein said host cell is E. coli.

41. A T cell receptor specific for a peptide according to claim 35.

42. A pharmaceutical composition comprising a peptide mixture according to claim 25, and a pharmaceutically acceptable carrier, diluent and/or excipient.

43. A pharmaceutical composition comprising a peptide according to the first or second peptide according to claim 25, and a pharmaceutically acceptable carrier, diluent and/or excipient.

44. A pharmaceutical composition comprising a peptide according to claim 30, and a pharmaceutically acceptable carrier, diluent and/or excipient.

45. A pharmaceutical composition comprising a T-cell mixture according to claim 33, and a pharmaceutically acceptable carrier, diluent and/or excipient.

46. A pharmaceutical composition comprising a T-cell preparation according to claim 34, and a pharmaceutically acceptable carrier, diluent and/or excipient.

47. A method of treating cancer, comprising administering a peptide mixture according to claim 25 or the first or second peptide thereof, a peptide according to claim 30, a T-cell mixture according to claim 33, a T-cell preparation according to claim 34, a nucleic acid molecule according to claim 35, or at least one nucleic acid molecule according to claim 36, to a patient in need thereof.

48. The method according to claim 47 further comprising, prior to the administering, identifying at least one GNAS mutation present in a sample taken from a patient, wherein the administering comprises administering to a patient in need thereof having at least one GNAS mutation.
